(19) United States
(12) Patent Application Publication
(10) Pub. No.: US 2025/0241354 A1
(43) Pub. Date: Jul. 31, 2025

(54) ORAL PRODUCTS

(71) Applicant: RAI STRATEGIC HOLDINGS, INC., Winston-Salem, NC (US)

(72) Inventors: Steven Lee Alderman, Lewisville, NC (US); Anthony Richard Gerardi, Winston-Salem, NC (US); Kristen Ann Spielbauer, Kernersville, NC (US); Michael F. Davis, Clemmons, NC (US); Michael Zawadzki, Durham, NC (US); Eric T. Hunt, Pfafftown, NC (US); Travis O'Neal, Pfafftown, NC (US)

(21) Appl. No.: 19/025,421

(22) Filed: Jan. 16, 2025

Related U.S. Application Data
(60) Provisional application No. 63/741,678, filed on Jan. 3, 2025.
(60) Provisional application No. 63/645,528, filed on May 10, 2024.
(60) Provisional application No. 63/621,910, filed on Jan. 17, 2024.

(51) Int. Cl. A24B 15/16; A24B 13/00; A24B 15/32; A24B 15/40

(52) U.S. Cl. CPC A24B 15/16; A24B 13/00; A24B 15/32; A24B 15/403

(57) ABSTRACT

The disclosure provides compositions configured for oral use, pouched products including such compositions, and methods of manufacturing the compositions and products. Example compositions configured for oral use include a filler component, water, and a 3-(1-methylpyrrolidin-2-yl)pyridine bearing one or more substituents on the pyridine ring.

### CROSS-REFERENCE TO RELATED APPLICATIONS

This application claims the benefit of U.S. Provisional Application No. 63/741,678, filed Jan. 3, 2025; and claims the benefit of U.S. Provisional Application No. 63/645,528, filed May 10, 2024; and claims the benefit of U.S. Provisional Application No. 63/621,910, Filed Jan. 17, 2024; all of which are herein incorporated by reference in their entirety.

### TECHNICAL FIELD

The present disclosure relates to an oral product. In particular, the present disclosure relates to products intended for human consumption. The products are configured for oral use and deliver substances such as flavors and/or active ingredients during use.

### BACKGROUND

There are many categories of products intended for oral use and enjoyment. For example, oral tobacco products containing nicotine, which is known to have both stimulant and anxiolytic properties, have been available for many years. Conventional formats for so-called “smokeless” tobacco products include moist snuff, snus, and chewing tobacco, which are typically formed almost entirely of particulate, granular, or shredded tobacco, and which are either portioned by the user or presented to the user in individual portions, such as in single-use pouches or sachets.

See for example, the types of smokeless tobacco formulations, ingredients, and processing methodologies set forth in U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; U.S. Pat. No. 7,810,507 to Dube et al.; U.S. Pat. No. 7,819,124 to Strickland et al.; U.S. Pat. No. 7,861,728 to Holton, Jr. et al.; U.S. Pat. No. 7,901,512 to Quinter et al.; U.S. Pat. No. 8,627,828 to Strickland et al.; U.S. Pat. No. 11,246,334 to Atchley, each of which is incorporated herein by reference. In addition, traditional tobacco materials and non-tobacco materials have been combined with other ingredients to form product formats distinct from traditional smokeless products, with example formats including lozenges, pastilles, gels, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

There is continuing interest in the development of new types of oral products that deliver advantageous sensorial or biological activity. Such products typically contain flavorants and/or active ingredients such as nicotine, caffeine, botanicals, or cannabidiol. The format of such products can vary and include pouched products containing a powdered or granular composition, lozenges, pastilles, liquids, gels, emulsions, meltable compositions, and the like. See, for example, the types of products described in US Patent App. Pub. Nos. 2022/0160675 to Gerardi et al.; 2022/0071984 to Poole et al.; 2021/0378948 to Gerardi et al.; 2021/0330590 to Hutchens et al.; 2021/0186081 to Gerardi et al.; 2021/0177754 to Keller et al; 2021/0177043 to Gerardi et al.; 2021/0177038 to Gerardi et al.; 2021/0169867 to Holton, Jr. et al.; 2021/0169792 to Holton, Jr. et al.; 2021/0169132 to Holton, Jr. et al.; 2021/0169121 to St. Charles, and 2021/0169122 to St. Charles, each of which is incorporated herein by reference.

### BRIEF SUMMARY

The present disclosure generally provides compositions configured for oral use and products comprising such compositions, along with a method of manufacturing such compositions and products. The compositions include a filler component, water, and a 3-(1-methylpyrrolidin-2-yl)pyridine bearing one or more substituents on the pyridine ring. The disclosure includes, without limitations, the following embodiments.

Embodiment 1: A composition configured for oral use, the composition comprising: A composition configured for oral use, the composition comprising: a filler component; water in an amount from about 1 to about 60% by weight, based on the total weight of the composition; and a substituted 3-(1-methylpyrrolidin-2-yl)pyridine having a structure according to Formula I:

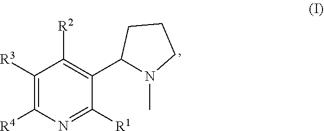

wherein R¹, R², R³, and R⁴ are each independently selected from the group consisting of hydrogen, alkyl, alkoxy, cycloalkyl, alkenyl, alkynyl, aryl, alkaryl, amino, halogen, and cyano, wherein any of said alkyl, alkoxy, cycloalkyl, alkenyl, alkenyl, alkynyl, aryl, alkaryl, and amino may optionally be substituted; and at least one of R¹, R², R³, and R⁴ are not hydrogen.

Embodiment 2: The composition of embodiment 1, wherein R¹, R², and R³ are each H.

Embodiment 3: The composition of embodiment 1 or 2, wherein R⁴ is optionally substituted C₁-C₆ alkyl, F, Cl, Br, OCH₃, OCH₂CH₃, or CN.

Embodiment 4: The composition of any one of embodiments 1-3, wherein R¹, R², and R³ are each H, and R⁴ is C₁-C₃ alkyl; preferably, wherein R⁴ is CH₃.

Embodiment 5: The composition of any one of embodiments 1-4, wherein the substituted 3-(1-methylpyrrolidin-2-yl)pyridine is racemic.

Embodiment 6: The composition of any one of embodiments 1-4, wherein the substituted 3-(1-methylpyrrolidin-2-yl)pyridine has an (R)-configuration or an (S)-configuration; preferably, wherein the substituted 3-(1-methylpyrrolidin-2-yl)pyridine has an (S)-configuration.

Embodiment 7: The composition of any one of embodiments 1-6, wherein the substituted 3-(1-methylpyrrolidin-2-yl)pyridine has a calculated log P of about 1 or greater, such as from about 1.2 to about 1.7.

Embodiment 8: The composition of any one of embodiments 1-7, wherein the composition is substantially free of 3-(1-methylpyrrolidin-2-yl)pyridine; optionally, wherein the composition is completely free of 3-(1-methylpyrrolidin-2-yl)pyridine.

Embodiment 9: The composition of any one of embodiments 1-8, wherein the composition is substantially free of acid components; optionally, wherein the composition is completely free of acid components.

Embodiment 10: The composition of any one of embodiments 1-8, further comprising an organic acid, an alkali metal salt of an organic acid, or a combination thereof, the organic acid having a log P value in a range from about 1 to about 12; optionally, wherein the organic acid has a log P value from about 3 to about 12, from about 3 to about 10, or from about 3 to about 8, and wherein at least a portion of the substituted 3-(1-methylpyrrolidin-2-yl)pyridine is present in the form of an ion pair with the organic acid, the alkali metal salt of an organic acid, or a combination thereof.

Embodiment 11: The composition of any one of embodiments 1-8, wherein at least a portion of the substituted 3-(1-methylpyrrolidin-2-yl)pyridine is present in the form of a salt, a co-crystal, or a salt co-crystal.

Embodiment 12: The composition of any one of embodiments 1-8, wherein at least a portion of the substituted 3-(1-methylpyrrolidin-2-yl)pyridine is bound to a polymeric resin.

Embodiment 13: The composition of any one of embodiments 1-12, wherein the substituted 3-(1-methylpyrrolidin-2-yl)pyridine is present in an amount from about 0.01 to about 10% by weight of the composition, calculated as the free base and based on the total weight of the composition.

Embodiment 14: The composition of any one of embodiments 1-13, wherein the filler component is cellulosic, optionally, wherein the filler component comprises microcrystalline cellulose.

Embodiment 15: The composition of any one of embodiments 1-14, further comprising one or more flavoring agents, one or more salts, one or more sweeteners, one or more binding agents, one or more humectants, one or more gums, or combinations thereof.

Embodiment 16: The composition of any one of embodiments 1-15, in the form of a gel, pastille, gum, chew, melt, tablet, lozenge, film, granular material, or powder.

Embodiment 17: The composition of any one of embodiments 1-15, enclosed in a pouch to form a pouched product, the composition optionally being in a granular form.

Embodiment 18: The composition of embodiment 17, having a moisture content from about 30 to about 60% by weight, based on the weight of the pouched product.

Embodiment 19: A composition configured for oral use, the composition comprising 2-methyl-5-(1-methylpyrrolidin-2-yl)pyridine in an amount from about 0.01 to about 10% by weight, based on the total weight of the composition; a filler component; and water in an amount from about 1 to about 60% by weight, or from about 30 to about 60% by weight, based on the total weight of the composition.

Embodiment 20: The composition of embodiment 19, wherein the filler component is cellulosic, optionally, wherein the filler component comprises microcrystalline cellulose.

Embodiment 21: The composition of embodiment 19, further comprising a buffer;
optionally, wherein the buffer is an alkali metal citrate.

Embodiment 22: The composition of any one of embodiments 1-21, wherein at least about 60% A of a total amount of 2-methyl-5-(1-methylpyrrolidin-2-yl)pyridine by weight present in the composition is released when said composition is placed in 500 mL of phosphate buffer at pH 7.4, at 37° C. for 50 minutes in a basket-type USP dissolution apparatus at a stirring speed of 50 RPM.

Embodiment 23: The composition of embodiment 22, wherein at least about 20% of a total amount of 2-methyl-5-(1-methylpyrrolidin-2-yl)pyridine by weight present in the composition is released at 10 minutes.

Embodiment 24: A method of preparing a composition configured for oral use comprising a substituted 3-(1-methylpyrrolidin-2-yl)pyridine, the method comprising: combining one or more fillers with one or more additional powder components, such as one or more salts and/or one or more sweeteners, to form a dry blend;
combining a dilute aqueous solution of substituted 3-(1-methylpyrrolidin-2-yl)pyridine with water and optional humectant to form a solution;
combining the solution with the dry blend to form a mixture;
mixing the resulting mixture to form a wet blend; and
optionally adding flavoring agent and/or water to the wet blend to form the composition.

Embodiment 25: The method of embodiment 22, wherein the substituted 3-(1-methylpyrrolidin-2-yl)pyridine is 2-methyl-5-(1-methylpyrrolidin-2-yl)pyridine.

These and other features, aspects, and advantages of the disclosure will be apparent from a reading of the following detailed description together with the accompanying drawings, which are briefly described below. The scope of the disclosure includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed embodiments should be viewed as intended to be combinable or separately/in isolation unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are examples only and should not be construed as limiting the disclosure.

FIG. 1 is a cross-sectional view of a non-limiting pouched product embodiment, taken across the width of the product, showing an outer pouch filled with a mixture of the present disclosure.

FIG. 2 is an illustration of a pouched product formed of a water-permeable fabric pouch having a composition therein and including an encapsulated payload according to an embodiment of the present disclosure.

FIG. 3 is an illustration of a pouched product formed of a water-permeable fabric pouch having a composition therein and including a plurality of capsules enclosing a payload according to an embodiment of the present disclosure.

FIG. 4 illustrates the steps of a method of preparing a composition configured for oral use according to a non-limiting embodiment of the present disclosure.

FIG. 5 is a graphical depiction of % release of active versus time according to non-limiting embodiments of the disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

The term “about” used throughout this specification is used to describe and account for small fluctuations. For example, the term “about” can refer to less than or equal to ±10%, such as less than or equal to ±5%, less than or equal to ±2%, less than or equal to ±1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

Reference to “dry weight percent” or “dry weight basis” refers to weight on the basis of dry ingredients (i.e., all ingredients except water). Reference to “wet weight” refers to the weight of the composition including water. Unless otherwise indicated, reference to “weight percent” of a composition reflects the total wet weight of the composition (i.e., including water).

### Composition Configured for Oral Use

The present disclosure provides compositions configured for oral use, products comprising such compositions, and methods of preparing the compositions. The compositions generally comprise a substituted 3-(1-methylpyrrolidin-2-yl)pyridine, a filler component, and water. The composition, products including such compositions, and the components of each thereof are further described herein below.

Substituted 3-(1-methylpyrrolidin-2-yl)pyridine

Disclosed herein are compositions configured for oral use comprising a substituted 3-(1-methylpyrrolidin-2-yl)pyridine. As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring. Compositions of the disclosure do not contain nicotine and do not contain any compounds obtained by chemical reactions utilizing nicotine as a starting material. In preferred embodiments, the substituted 3-(1-methylpyrrolidin-2-yl)pyridine present is not synthetically derived from nicotine.

In some embodiments, the compositions and products of the disclosure can be characterized as completely free or substantially free of nicotine (3-(1-methylpyrrolidin-2-yl)pyridine). By “substantially free” it is meant that no nicotine has been intentionally added, beyond trace amounts that may be present e.g., as an impurity in another component, including as a minor impurity in the substituted 3-(1-methylpyrrolidin-2-yl)pyridine. For example, some embodiments can be characterized as having less than 0.01% by weight of nicotine, or less than 0.001% by weight of nicotine, or less than 0.0001%, or even 0% by weight of nicotine, calculated as the free base and based on the total weight of the composition. In some embodiments the composition is completely free of (R)-, (S)-, and (R/S)-3-(1-methylpyrrolidin-2-yl)pyridine (e.g., having 0% by weight of nicotine, including racemic nicotine and nicotine enantiomers, calculated as the free base and based on the total weight of the composition).

In some embodiments, the substituted 3-(1-methylpyrrolidin-2-yl)pyridine has a structure according to Formula I:

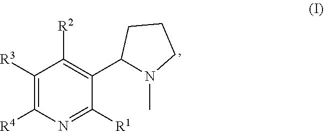

wherein:
   R¹, R², R³, and R⁴ are each independently selected from the group consisting of hydrogen, alkyl, alkoxy, cycloalkyl, alkenyl, alkynyl, aryl, alkaryl, amino, halogen, and cyano, wherein any of said alkyl, alkoxy, cycloalkyl, alkenyl, alkenyl, alkynyl, aryl, alkaryl, and amino may optionally be substituted; and at least one of R¹, R², R³, and R⁴ are not hydrogen.

Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, R¹, R², and R⁴ are each H, and R³ is a non-hydrogen substituent.

In some embodiments, R³ is optionally substituted C₁-C₆ alkyl, F, Cl, Br, OMe, OEt, or CN. In some embodiments, R³ is Me or Et. n some embodiments, R³ is F.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C₁-C₆ alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is CH₃. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; ₇₆₁ TSRC Conference 2023, Norfolk, VA, USA).

Without wishing to be bound by any particular theory, it is believed that certain substitutions for hydrogen on the pyridine ring of the substituted 3-(1-methylpyrrolidin-2-yl)pyridine compound preserve the general pharmacological profile and physiological effects of nicotine while offering the potential for one or more of greater potency, reduced product consumption, more rapid and/or complete absorption, and the like. Particularly, it is believed that in some embodiments, substituted 3-(1-methylpyrrolidin-2-yl)pyridines of the disclosure are readily absorbed through oral mucosa by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The substituted 3-(1-methylpyrrolidin-2-yl)pyridine (e.g., 2-methyl-5-(1-methylpyrrolidin-2-yl)pyridine) may be present as a single enantiomer or as a mixture of enantiomers. In some embodiments, the substituted 3-(1-methylpyrrolidin-2-yl)pyridine is present in racemic form, meaning there are equal amounts of (R)- and (S)-enantiomers present. In some embodiments, the composition comprises unequal amounts of (R)- and (S)-enantiomer (i.e., is enriched in either the (R)- or (S)-enantiomer. In some embodiments, the composition predominantly comprises the substituted 3-(1-methylpyrrolidin-2-yl)pyridine (e.g., 2-methyl-5-(1-methylpyrrolidin-2-yl)pyridine) in the (R)-configuration, for example, about 90% or more of the total quantity of substituted 3-(1-methylpyrrolidin-2-yl)pyridine present is in the (R)-configuration. In some embodiments, the composition predominantly comprises the substituted 3-(1-methylpyrrolidin-2-yl)pyridine (e.g., 2-methyl-5-(1-methylpyrrolidin-2-yl)pyridine) in the (S)-configuration, for example, about 90% or more of the total quantity of substituted 3-(1-methylpyrrolidin-2-yl)pyridine present is in the (S)-configuration. In some embodiments, the composition comprises 95% or more of the (S)-configuration of the substituted 3-(1-methylpyrrolidin-2-yl)pyridine (e.g., 2-methyl-5-(1-methylpyrrolidin-2-yl)pyridine), based on the total amount of substituted 3-(1-methylpyrrolidin-2-yl)pyridine present.

The quantity of substituted 3-(1-methylpyrrolidin-2-yl)pyridine (e.g., 2-methyl-5-(1-methylpyrrolidin-2-yl)pyridine) present in the composition may vary. Typically, the substituted 3-(1-methylpyrrolidin-2-yl)pyridine (e.g., 2-methyl-5-(1-methylpyrrolidin-2-yl)pyridine, calculated as the free base) is present in a concentration of at least about 0.001% by weight of the composition, such as in a range from about 0.01% to about 10%. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition. In some embodiments, the composition comprises 2-methyl-5-(1-methylpyrrolidin-2-yl)pyridine in an amount from about 0.01 to about 10% by weight, based on the total weight of the composition.

The substituted 3-(1-methylpyrrolidin-2-yl)pyridine (e.g., 2-methyl-5-(1-methylpyrrolidin-2-yl)pyridine) may be present in the composition as the free base, as a salt with a suitable acid, as a cocrystal with a suitable coformer, or in the form of an ion pair with a suitable organic acid. Each of these forms is described further herein below.

### Free Base

In some embodiments, the substituted 3-(1-methylpyrrolidin-2-yl)pyridine exhibits sufficient stability, aqueous solubility, and oral bioavailability such that the free base is suitable for inclusion in the composition. Accordingly, in some embodiments, the substituted 3-(1-methylpyrrolidin-2-yl)pyridine is present substantially or completely as the free base. In such embodiments, one of skill in the art will recognize that the composition is substantially free of acidic components. By “substantially free” it is meant that no acidic component (e.g., inorganic acid, organic acid, or acids capable of salt, ion pair, or co-crystal formation) has been intentionally added, beyond trace amounts that may be present e.g., as an impurity in another component. For example, some embodiments can be characterized as having less than 0.001% by weight of any acid component, or less than 0.0001%, or even 0% by weight of any acid component, based on the total weight of the composition. In some embodiments, the composition is completely free of any acid component (i.e., characterized as 0% or as having an amount below the limit of detection). In some embodiments, the substituted 3-(1-methylpyrrolidin-2-yl)pyridine is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine can be employed in the form of a salt. A “salt” of a substituted 3-(1-methylpyrrolidin-2-yl)pyridine is a form characterized by interaction between the substituted 3-(1-methylpyrrolidin-2-yl)pyridine in ionic form and a coformer in ionic form (e.g., an acid) via the transfer of one or more protons from the coformer donor to the nicotine acceptor. The structure of substituted 3-(1-methylpyrrolidin-2-yl)pyridines as disclosed herein are such that they comprise two nitrogen atoms that are capable of accepting protons from a coformer and, accordingly, it can be present in non-protonated, mono-protonated, and/or di-protonated form in a given sample. Salts of substituted 3-(1-methylpyrrolidin-2-yl)pyridines can be provided using the types of ingredients and techniques set forth for nicotine in U.S. Pat. No. 2,033,909 to Cox et al. and Perfetti, Beitrage Tabakforschung Int., 12: 43-54 (1983), which are incorporated herein by reference. Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt).

In some embodiments, a hydrophilic acid is chosen so as to increase water solubility and/or decrease lipophilicity of the salt. Lipophilicity of a substituted 3-(1-methylpyrrolidin-2-yl)pyridine salt can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments.

Accordingly, in some embodiments, the substituted 3-(1-methylpyrrolidin-2-yl)pyridine salt has a log D from about −1.0 to about 3 at a pH in a range from about 3 to about 11, such as from about −0.5 to about 2, about −0.3 to about 1, or about −0.1 to about 0.

In some embodiments, the substituted 3-(1-methylpyrrolidin-2-yl)pyridine is present in the form of a salt with tartaric acid, succinic acid, orotic acid, fumaric acid, pyroglutamic acid, or galactaric acid.

The stoichiometry of the salts as described herein can vary. For example, in some embodiments, the stoichiometry can range from about 5:1 to about 1:5 substituted 3-(1-methylpyrrolidin-2-yl)pyridine:acid. In some embodiments, the ratio of substituted 3-(1-methylpyrrolidin-2-yl)pyridine to acid is 2:1, 1:1, or 1:2. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing polymorphism) or from the existence of different conformers of the same molecule (conformational polymorphism). Pseudopolymorphism is the result of hydration or solvation of a material and is also referred to as solvomorphism,

### Resin Complex

In some embodiments, at least a portion of the substituted 3-(1-methylpyrrolidin-2-yl)pyridine may be present in the form of a polymer complex, where the substituted 3-(1-methylpyrrolidin-2-yl)pyridine is bound to an acidic polymer. The polymer of a substituted 3-(1-methylpyrrolidin-2-yl)pyridine-polymer complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of substituted 3-(1-methylpyrrolidin-2-yl)pyridine complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine is present in the form of a polymer complex, the composition further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine may be present in the form of a co-crystal. A “co-crystal” of a substituted 3-(1-methylpyrrolidin-2-yl)pyridine is a form of comprising a substituted 3-(1-methylpyrrolidin-2-yl)pyridine as described herein and at least one other component (“coformer”), both in neutral form. Specifically, as defined in a United States Food and Drug Administration industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., the substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-.1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo4-hydroxybenzoic acid, 3-bromo-5-hydroxybenzoic acid. 4-bromo*-2*-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-*hydroxybenzoic acid, 4-fluoro-3-hydroxybenzoic acid, 2-fluoro-4-hydroxybenzoic acid, 5-fluoro-2-hydroxybenzoic acid. 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3- formyl-4-hydroxybenzoic acid, 5-(acetylamino)-2-hydroxybenzoic acid), 4-nitro-2-hydroxybenzoic acid, 3,5)-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid. 3,5-diodo-2-hydroxybenzoic acid, 4-amino-5-chloro-2-hydroxybenzoic acid, 3,5-dinitro-2-hydroxybenzoic acid, 2,46-tribromo-2-hydroxybenzoic acid, 2,3,5,6-tetrafluoro-4-hydroxybenzoic acid, and 23,4,5-tetrafluoro-*6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (f-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihydroxybenzoic acid (orsellinic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro 2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid), Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexadiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine is present in the form of a salt co-crystal. A “salt co-crystal” is a type of hybrid structure with both salt and co-crystal characteristics. Typically, a substituted 3-(1-methylpyrrolidin-2 yl)pyridine molecule within a salt co-crystal is associated with at least two coformers (which may be the same or different), wherein one coformer is in ionic form (e.g., an acid) and transfers a proton to the substituted 3-(1-methylpyrrolidin-2-yl)pyridine molecule and wherein a second coformer does not transfer a proton to the substituted 3-1-methylpyrrolidin-2-yl)pyridine molecule, Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 substituted 3-(1-methylpyrrolidin-2-yl)pyridine:coformer. Where more than one coformer is used to form a co-crystal or salt co-crystal, the ratios of the coformers with respect to both the substituted 3-(1-methylpyrrolidin-2-yl)pyridine and to one another can also vary,

The co-crystals and salt co-crystals described herein can, in some embodiments, exist in various polymorphic and pseudopolymorphic forms, as well as solvates and hydrates,

In some embodiments, the substituted 3-(1-methylpyrrolidin-2-yl)pyridine is present in the form of a salt-co-crystal. In some embodiments, the salt-co-crystal is a bis-orotic acid salt-co-crystal. In some embodiments, the bis-orotic acid salt-co-crystal is a hemi-hydrate, Ion pairing

In some embodiments, at least a portion of the substituted 3-(1-methylpyrrolidin-2-yl)pyridine is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (i.e., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the composition as disclosed herein, and/or upon oral use of said composition (e.g., upon contact with saliva), the substituted 3-(1-methylpyrrolidin-2-yl)pyridine and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed composition, both before and during use by the consumer, may vary based on, for example, pH, the nature of the acid, the concentration of substituted 3-(1-methylpyrrolidin-2-yl)pyridine, the concentration of the acid or conjugate base of the acid present in the composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine between octanol and water, or by performing membrane permeation studies of aqueous solutions of the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine (e.g., 2-methyl-5-(1-methylpyrrolidin-2-yl)pyridine) through the oral mucosa. However, as described above, in some embodiments, the properties of the substituted 3-(1-methylpyrrolidin-2-yl)pyridine (e.g., 2-methyl-5-(1-methylpyrrolidin-2-yl)pyridine) are such that no ion pairing is required, and accordingly, the composition is substantially or completely free of any ion pairing. By “substantially free” it is meant that no measurable degree of ion pairing is present.

In embodiments where ion pairing with the substituted 3-(1-methylpyrrolidin-2-yl)pyridine are desired, the composition comprises an organic acid, an alkali metal salt of an organic acid, or both. In such embodiments, at least a portion of the substituted 3-(1-methylpyrrolidin-2-yl)pyridine is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the composition of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the composition may be characterized as substantially or completely free of organic acids (i.e., having less than 0.001% by weight of organic acid, or less than 0.0001%, or even 0% by weight of organic acid, based on the total weight of the composition, or as having an amount of organic acid below the limit of detection). This is not to be interpreted as meaning that the composition is substantially or completely free of substituted 3-(1-methylpyrrolidin-2-yl)pyridine salts or substituted 3-(1-methylpyrrolidin-2-yl)pyridine co-crystals unless explicitly recited.

### Organic Acid

In embodiments where ion pairing with the substituted 3-(1-methylpyrrolidin-2-yl)pyridine are desired, the composition comprises an organic acid as defined herein above, and/or an alkali metal salt thereof. Suitable organic acids for ion pairing will typically have a range of lipophilicities (i.e., a polarity giving an appropriate balance of water and organic solubility). Typically, lipophilicities of suitable organic acids, as indicated by log P, will vary between about 0 and about 12 (with values above 0 indicating higher solubility in octanol than in water). In some embodiments, the organic acid has a log P value from about −0.05 to about 1, e.g., from about −0.05, about 0.1, about 0.2, about 0.3, about 0.4, or about 0.5, to about 0.6, about 0.7, about 0.8, or about 0.9, or about 1.0, In some embodiments, the organic acid has a log P value from about 1 to about 12, e.g., from about 1.0. about 1.5, about 2.0, about 2.5, about 3.0, about 3.5, about 4.0, about 4.5, about 5.0, about 5.5, about 6.0, about 6.5, about 7.0, about 7.5, or about 8.0, to about 8.5, about 9.0, about 9.5, about 10.0, about 10.5, about 11.0, about 11.5, or about 12.0.

Without wishing to be bound by theory, it is believed that moderately lipophilic organic acids (e.g., log P of from about 1.4 to about 4.5) produce ion pairs with a substituted 3-(1-methylpyrrolidin-2-yl)pyridine which are of a polarity providing good octanol-water partitioning of the ion pair, and hence partitioning of substituted 3-(1-methylpyrrolidin-2-yl)pyridine, into octanol versus water. As discussed above, such partitioning into octanol is predictive of favorable oral availability.

In specific embodiments, the organic acid for ion pairing has a log P value from about 3.0 to about 8.0, about 10.0, or even 12.0. In some embodiments, the presence of certain solvents or solubilizing agents (e.g., inclusion in the composition of glycerin or propylene glycol) may be beneficial in solubilizing organic acids and the corresponding salts or ion pairs thereof with the substituted 3-(1-methylpyrrolidin-2-yl)pyridine for highly lipophilic organic acids (e.g., higher than about 4.5).

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C (═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid for ion pairing is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid for ion pairing is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid for ion pairing may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid for ion pairing may include more than one carboxylic acid group and one or more hydroxyl groups. Non-limiting examples of such acids include tartaric acid, citric acid, and the like.

In some embodiments, the organic acid for ion pairing is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful for ion pairing in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-1b-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids for ion pairing include, but are not limited to, the list of organic acids in Table 1.

TABLE 1
Non-limiting examples of suitable organic acids for ion pairing

| Acid Name | log(P)* |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-(4-Isobutylphenyl)propanoic acid | 3.5 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptanesulfonic acid | 2.0 |
| 1-octanesulfonic acid | 2.5 |
| 1-nonanesulfonic acid | 3.1 |
| monooctyl succinate | 2.8 |
| tocopherol succinate | 10.2 |
| monomenthyl succinate | 3 |
| monomenthyl glutarate | 3.4 |
| norbixin ((2E,4E,6E,8E,10E,12E,14E,16E,18E)-4,8,13,17- | 7.2 |
| tetramethylicosa-2,4,6,8,10,12,14,16,18-nonaenedioic | |
| acid) | |
| bixin ((2E,4E,6E,8E,10E,12E,14E,16Z,18E)-20-methoxy- | 7.5 |
| 4,8,13,17-tetramethyl-20-oxoicosa-2,4,6,8,10,12,14,16,18- | |
| nonaenoic acid) | |
| nicotinic acid | 0.4 |
| propanoic acid | 0.3 |
| 3-hydroxybutanoic acid | −0.2 |
| pimelic acid | 1.1 |
| succinic acid | −0.1 |
| salicylic acid | 2.3 |

*Values obtained from PubChem or calculated

The selection of organic acid for ion pairing may further depend on additional properties in addition to consideration of the log P value. For example, an organic acid should be one recognized as safe for human consumption, and which has acceptable flavor, odor, volatility, stability, and the like. Determination of appropriate organic acids is within the purview of one of skill in the art.

In some embodiments, the organic acid for ion pairing is a mono ester of a dicarboxylic acid or a poly-carboxylic acid. In some embodiments, the dicarboxylic acid is malonic acid, succinic acid, glutaric acid, adipic acid, fumaric acid, maleic acid, or a combination thereof. In some embodiments, the dicarboxylic acid is succinic acid, glutaric acid, fumaric acid, maleic acid, or a combination thereof. In some embodiments, the dicarboxylic acid is succinic acid, glutaric acid, or a combination thereof.

In some embodiments, the alcohol forming the mono ester of the dicarboxylic acid is a lipophilic alcohol. Examples of suitable lipophilic alcohols include, but are not limited to, octanol, menthol, and tocopherol. In some embodiments, the organic acid is an octyl mono ester of a dicarboxylic acid, such as monooctyl succinate, monooctyl fumarate, or the like. In some embodiments, the organic acid is a monomenthyl ester of a dicarboxylic acid. Certain menthyl esters may be desirable in oral compositions as described herein by virtue of the cooling sensation they may provide upon use of the product comprising the composition. In some embodiments, the organic acid is monomenthyl succinate, monomenthyl fumarate, monomenthyl glutarate, or a combination thereof. In some embodiments, the organic acid is a monotocopheryl ester of a dicarboxylic acid. Certain tocopheryl esters may be desirable in oral compositions as described herein by virtue of the antioxidant effects they may provide. In some embodiments, the organic acid is tocopheryl succinate, tocopheryl fumarate, tocopheryl glutarate, or a combination thereof.

In some embodiments, the organic acid for ion pairing is a carotenoid derivative having one or more carboxylic acids. Carotenoids are tetraterpenes, meaning that they are produced from 8 isoprene molecules and contain 40 carbon atoms. Accordingly, they are usually lipophilic due to the presence of long unsaturated aliphatic chains, and are generally yellow, orange, or red in color. Certain carotenoid derivatives can be advantageous in oral compositions by virtue of providing both ion pairing and serving as a colorant in the composition. In some embodiments, the organic acid is 2E,4E,6E,8E,10E,12E,14E,16Z,18E)-20-methoxy-4,8,13,17-tetramethyl-20-oxoicosa-2,4,6,8,10,12,14,16,18-nonaenoic acid (bixin) or an isomer thereof. Bixin is an apocarotenoid found in annatto seeds from the achiote tree (Bixa orellana) and is the naturally occurring pigment providing the reddish orange color to annatto. Bixin is soluble in fats and alcohols but insoluble in water, and is chemically unstable when isolated, converting via isomerization into the double bond isomer, trans-bixin (0-bixin), having the structure:

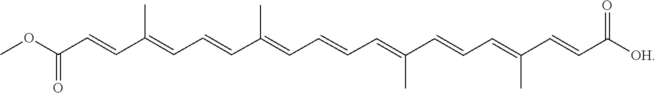 [SMILES: COC(=O)/C=C/C(C)=C/C=C/C(C)=C/C=C/C=C(C)/C=C/C=C(C)/C=C/C(=O)O]

In some embodiments, the organic acid for ion pairing is (2E,4E,6E,8E,10E,12E,14E,16E,18E)-4,8,13,17-tetramethylicosa-2,4,6,8,10,12,14,16,18-nonaenedioic acid (norbixin), a water-soluble hydrolysis product of bixin having the structure:

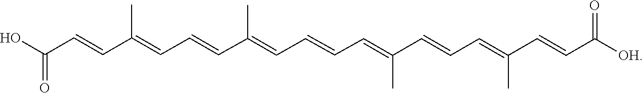 [SMILES: CC(/C=C/C=C(C)/C=C/C(=O)O)=C\C=C\C=C(C)\C=C\C=C(C)\C=C\C(=O)O]

In some embodiments, the organic acid for ion pairing is selected from the group consisting of nicotinic acid, propanoic acid, 3-hydroxybutanoic acid, pimelic acid, and succinic acid. In some embodiments, the organic acid for ion pairing is benzoic acid.

In some embodiments, more than one organic acid for ion pairing may be present. For example, the composition may comprise two, or three, or four, or more organic acids for ion pairing. Accordingly, reference herein to “an organic acid” contemplates mixtures of two or more organic acids. The relative amounts of the multiple organic acids may vary. For example, a composition may comprise equal amounts of two, or three, or more organic acids, or may comprise different relative amounts. In this manner, it is possible to include certain organic acids (e.g., citric acid or myristic acid) which have a log P value outside the desired range, when combined with other organic acids to provide the desired average log P range for the combination. In some embodiments, it may be desirable to include organic acids in the composition for ion pairing which have log P values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the composition remains below the threshold which would be found objectionable from a sensory perspective.

In some embodiments, the composition comprises an organic acid for ion pairing which is a monoester of a dicarboxylic acid or is a carotenoid derivative having one or more carboxylic acids as described herein above, and further comprises an additional organic acid or salt thereof.

In some embodiments, the additional organic acid is benzoic acid, an alkali metal salt thereof, or a combination thereof.

In some embodiments, the composition comprises an alkali metal salt of an organic acid. For example, at least a portion of the organic acid may be present in the composition in the form of an alkali metal salt. Suitable alkali metal salts include lithium, sodium, and potassium. In some embodiments, the alkali metal is sodium or potassium. In some embodiments, the alkali metal is sodium. In some embodiments, the composition comprises an organic acid and a sodium salt of the organic acid.

In some embodiments, the molar ratio of the organic acid to the sodium salt (or other alkali metal) of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the composition, wherein the organic acid is added in excess of the sodium salt, in equimolar quantities with the sodium salt, or as a fraction of the sodium salt. One of skill in the art will recognize that the relative amounts will be determined by the desired pH of the composition, as well as the desired ionic strength. For example, the organic acid may be added in a quantity to provide a desired pH level of the composition, while the alkali metal (e.g., sodium) salt is added in a quantity to provide the desired extent of ion pairing. As one of skill in the art will understand, the quantity of organic acid (i.e., the protonated form) present in the composition, relative to the alkali metal salt or conjugate base form present in the composition, will vary according to the pH of the composition and the pKa of the organic acid, as well as according to the actual relative quantities initially added to the composition.

The amount of organic acid or alkali metal salt thereof present in the composition, relative to the substituted 3-(1-methylpyrrolidin-2-yl)pyridine (e.g., 2-methyl-5-(1-methylpyrrolidin-2-yl)pyridine), may vary. Generally, as the concentration of the organic acid (or the conjugate base thereof) increases, the percent of substituted 3-(1-methylpyrrolidin-2-yl)pyridine that is ion paired with the organic acid increases. This typically increases the partitioning of the substituted 3-(1-methylpyrrolidin-2-yl)pyridine, in the form of an ion pair, into octanol versus water as measured by the log P (the log₁₀ of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, calculated as the free base of the substituted 3-(1-methylpyrrolidin-2-yl)pyridine.

In some embodiments, the composition comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, on a free-base basis. In some embodiments, the organic acid, the alkali metal salt thereof, or the combination thereof, is present in a molar ratio with the substituted 3-(1-methylpyrrolidin-2-yl)pyridine from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the composition comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine. In some embodiments, the molar ratio of the total amount of benzoate to the substituted 3-(1-methylpyrrolidin-2-yl)pyridine is about 3.2 or about 4.8.

In some embodiments, the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. Reference herein to “a composition pH” means the pH of an aqueous solution of the composition prepared by dissolving or suspending 5 grams of composition in 95 grams of water and measuring the pH of the resulting solution with a calibrated pH meter.

In some embodiments, the organic acid inclusion is sufficient to provide a composition pH of from about 4.5 to about 6.5, for example, from about 4.5, about 5.0, or about 5.5, to about 6.0, or about 6.5. In some embodiments, the desired composition pH is from about 4.5 to about 6.5, and the organic acid is provided in a quantity sufficient to provide such a pH. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the composition to the desired value. In some embodiments, a buffer (e.g., a buffer as described herein below) is added to the composition to the desired value, and/or to maintain the pH of the composition at the desired value.

In some embodiments, the oral composition further comprises a solubility enhancer to increase the solubility of one or more of the organic acid or salt thereof. Suitable solubility enhancers include, but are not limited to, humectants as described herein, such as glycerol or propylene glycol.

### Additional Active Agents

In some embodiments, the oral composition further comprises one or more additional active agents, such as caffeine, theanine, cannabinoids, an herbal extract, and agonists or allosteric modulators of the a7 nicotinic receptor subtype (α7 nAChR).

In some embodiments, the oral composition further comprises caffeine, theanine, or a combination thereof.

In some embodiments, the oral composition further comprises a cannabinoid.

In some embodiments, the oral composition further comprises an herbal extract.

In some embodiments, the oral composition further comprises an agonist of the α7 nAChR.

In some embodiments, the agonist of the α7 nAChR is pentylsedinine, an alkaloid present in Lobelia species. See, e.g., Nat Prod Commun. 2015 August; 10(8):1355-7. In some embodiments, the agonist of the α7 nAChR is lobeline or a related natural product from the Lobelia species. In some embodiments, the agonist of the α7 nAChR is lobeline. In some embodiments, the agonist of the α7 nAChR is lobelanidine. In some embodiments, the agonist of the α7 nAChR is a Lobelia sp. extract. See, for example, Front. Pharmacol. 7 Mar. 2021, Sec. Ethnopharmacology, Volume 12-2021, which describes lobeline and other natural products present in extracts of Lobelia species. In some embodiments, the agonist of the α7 nAChR is 3-(2,4-dimethoxybenzylidene)-anabaseine (GTS-21). See, for example, Briggs et al, Pharmacology, Biochemistry, and Behavior 1997, 57 (1-2): 231-241.

In some embodiments, the agonist of the α7 nAChR is citicoline (cytidine-5-diphosphocholine). In some embodiments, the agonist of the α7 nAChR is alpha-GPC (L-alpha glycerylphosphorylcholine).

In some embodiments, the agonist of the α7 nAChR is 5-hydroxyindole.

In some embodiments, the oral composition further comprises an allosteric modulator of the α7 nAChR. In some embodiments, the allosteric modulator is galantamine. In addition to activity as an acetylcholinesterase inhibitor, galantamine has been reported to act as an allosteric modulator of the α7 nAChR. See, e.g., Drugs.com. Aug. 8, 2023.

The amount of additional active agent can vary, depending on the type of product and desired characteristics thereof. In some embodiments, the composition comprises one or more additional active agents in an amount from about 0.01 to about 30% by weight, based on the total weight of the composition.

### Filler

The composition as described herein comprises one or more fillers. Fillers may fulfill multiple functions, such as enhancing certain organoleptic properties such as texture and mouthfeel, enhancing cohesiveness or compressibility of the product, and the like. Generally, fillers are porous particulate materials and may be inorganic or organic (e.g., cellulose-based). For example, suitable organic fillers are any non-tobacco plant material or derivative thereof, including cellulose materials derived from such sources. Additional examples of organic fillers include maltodextrin, dextrose, lactose, and sugar alcohols. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Combinations of fillers can also be used, such as multiple organic fillers, inorganic fillers, or combinations of organic and inorganic fillers.

In some embodiments, the filler comprises or is a cellulose material or cellulose derivative. One particularly suitable filler for use in the compositions described herein is microcrystalline cellulose (“mcc”). The mcc may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In some embodiments, the composition comprises mcc as the particulate filler. The quantity of mcc present may vary according to the desired properties.

In some embodiments, the filler comprises a starch or is derived from a starch. The term “starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications and are considered to be “modified” starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold-water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate. In some embodiments, the filler comprises or is a mixture of glucose and starch-derived polysaccharides. One such suitable mixture of glucose and starch-derived polysaccharides is EMDEX®, available from JRS PHARMA LP, USA, 2981 Route 22, Patterson, NY 12563-2359.

In some embodiments, the filler comprises or is an inorganic material. Examples of potential inorganic fillers include calcium carbonate, calcium phosphate, and bioceramic materials (e.g., porous hydroxyapatite).

In certain embodiments of the present disclosure, a substantially spherical filler in particulate form is utilized, and such fillers can be defined by their sphericity, which is a measure of how closely an object resembles a perfect sphere. Sphericity (Ψ) can be measuring using the equation below, wherein Vₚ is the volume of the object and Aₚ is the surface area of the object.

Ψ=(π^{1/3}(6Vₚ)^{2/3})/Aₚ

The sphericity of a sphere is unity by definition and any shape that is not a perfect sphere will have a sphericity less than 1. In certain embodiments, the sphericity of the substantially spherical fillers of the present disclosure will be about 0.7 or higher, such as about 0.8 or higher or about 0.9 or higher (e.g., about 0.7 to 1 or about 0.75 to 1 or about 0.8 to 1 or about 0.85 to 1, or about 0.9 to 1).

The average diameter (mean) or D50 (median) particle size of the substantially spherical particulate filler particles provided herein can vary, and is not particularly limited. For example, in some embodiments, the spherical filler particles have an average diameter and/or a D50 value of about 100 μm to about 2000 μm, such as about 250 μm to about 750 μm. For example, in some embodiments, the average diameter is about 100 μm to about 500 μm, e.g., about 100 μm to about 400 μm, about 100 μm to about 300 μm, about 100 μm to about 200 μm, about 200 μm to about 500 μm, about 200 μm to about 400 μm, about 200 μm to about 300 μm, about 300 μm to about 500 μm, about 300 μm to about 400 μm, or about 400 μm to about 500 μm. In some embodiments, the average diameter is about 500 μm to about 1000 μm, e.g., about 500 μm to about 900 μm, about 500 μm to about 800 μm, about 500 μm to about 700 μm, about 500 μm to about 600 μm, about 600 μm to about 1000 μm, about 600 μm to about 900 μm, about 600 μm to about 800 μm, about 600 μm to about 700 μm, about 700 μm to about 1000 μm, about 700 μm to about 900 μm, about 700 μm to about 800 μm, about 800 μm to about 1000 μm, about 800 μm to about 900 μm, or about 900 μm to about 1000 μm. In some embodiments, the substantially spherical filler component has an average diameter and/or D50 value of about 300 to 650 microns, such as about 350 to about 500 microns.

The distribution of diameters around this average diameter (i.e., the particle size distribution) can also vary; in some embodiments, the distribution of diameters is close to the listed value (e.g., +/− about 25% of the stated value, +/− about 20% of the stated value, +/− about 15% of the stated value, +/− about 10% of the stated value, +/− about 5% of the stated value, or +/− about 1% of the stated value. The disclosure is not, however, limited to materials with such narrow distributions; in other embodiments, the diameter of the MCC spheres within a given material can vary within a wider range. Particle size distributions can be determined using a sieve analysis.

In some embodiments, the substantially spherical filler comprises microcrystalline cellulose (“MCC”). The MCC may be synthetic or semi-synthetic, or it may be obtained entirely from natural celluloses. By “substantially spherical MCC” is meant a material comprising, consisting essentially of, or consisting of MCC, wherein the material is a substantially spherical particulate filler component as referenced herein above.

The diameter of the substantially spherical MCC particles can vary within the scope of the disclosure, e.g., according to the values disclosed herein with respect to substantially spherical particulate filler components generally. Various commercial suppliers provide MCC spheres in designated diameter sizes and such commercial materials can be used in certain embodiments according to the present disclosure. Examples of suitable MCC spheres include, but are not limited to, Vivapur® MCC spheres from JRS Pharma, available, e.g., with particle sizes of 100-200 μm (Vivapur® 100), 200-355 μm (Vivapur® 200), 355-500 μm (Vivapur® 350), 500-710 μm (Vivapur® 500), 710-1000 μm (Vivapur®700), and 1000-1400 μm (Vivapur® 1000). Further examples of suitable MCC spheres include, but are not limited to, Celphere™ MCC spheres from Asahi Kasei Corporation, available, e.g., with particle sizes of 75-212 μm (Celphere™ SCP-100), 106-212 m (Celphere™ CP-102), 150-300 μm (Celphere™ CP-203), 300-500 μm (Celphere™ CP-305), and 500-710 μm (Celphere™ CP-507). It is noted that this application focuses on the use of substantially spherical MCC; however, this disclosure is not meant to be limiting. Other types of materials that can serve the function of a filler as provided herein and which can be provided in substantially spherical form can be used in place of (or in addition) to the exemplified substantially spherical MCC filler without deviating from the scope of the disclosure.

Advantageously, the use of a filler in substantially spherical particulate form can endow the composition and product into which it is incorporated with unique physical properties. For example, in some embodiments, the replacement of at least a portion of conventional particulate filler within a pouched composition with substantially spherical particulate filler can allow for alternative moisture levels within the pouched composition as compared with compositions comprising all conventional particulate filler. For example, in some embodiments, use of the disclosed substantially spherical filler can provide for a drier pouch than typically feasible using conventional fillers (e.g., all other components and amounts thereof being roughly equivalent). For example, in some embodiments, the disclosed composition, comprising a substantially spherical particulate filler, can provide a pouched product having a moisture content/oven volatile content of about 32% or less, about 30% or less, about 28% or less, about 27% or less, about 26% or less, about 25% or less, about 24% or less, about 23% or less, about 22% or less, about 21% or less, about 20% or less, about 19% or less, about 18% or less, about 17% or less, about 16% or less, or about 15% or less. In some embodiments, the moisture content of the disclosed compositions can be about 5% to about 32%, e.g., about 5% to about 25%, about 5% to about 20%, about 5% to about 15%, about 10% to about 25%, about 10% to about 20%, or about 10% to about 15%. Moisture/oven volatile content can be measured by drying a sample in an oven and precisely measuring the weight of the sample before and after drying so that the difference gives the amount of water or other oven volatiles evaporated.

In some embodiments, the provision of a drier pouch (e.g., about 32% moisture or less, as referenced in the prior paragraph) due to the inclusion of particulate filler in the form of a substantially spherical particulate filler can allow for different components to be suitably contained within the pouch as compared with inclusion of particulate filler in conventional form. For example, certain components that may desirably be included within a pouched composition may be more feasibly incorporated within a drier composition (e.g., where such components are water-soluble or water-reactive). For example, certain components encapsulated with a water-soluble exterior may more advantageously be included within a drier composition/product (e.g., a composition/product having a moisture content within the ranges referenced herein).

In some embodiments, the replacement of at least a portion of conventional particulate filler within a pouched composition with substantially spherical particulate filler can provide for modified release rates of substituted 3-(1-methylpyrrolidin-2-yl)pyridine relative to compositions comprising all conventional particulate filler. At the same moisture level, a substantially spherical MCC will generally release a substituted 3-(1-methylpyrrolidin-2-yl)pyridine of the disclosure faster than conventional particulate MCC. Similarly, in some embodiments, at the same moisture level, a substantially spherical particulate filler will release a flavorant faster than a conventional particulate filler.

The amount of filler can vary but is typically up to about 75 percent of the composition by weight, based on the total weight of the composition. A typical range of filler (e.g., MCC, including spherical MCC) within the composition can be from about 10 to about 75 percent by total weight of the composition, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of filler is at least about 10 percent by weight, such as at least about 20 percent, or at least about 25 percent, or at least about 30 percent, or at least about 35 percent, or at least about 40 percent, based on the total weight of the composition.

In some embodiments, the filler further comprises a cellulose derivative or a combination of such derivatives. In some embodiments, the composition comprises from about 1 to about 10% of the cellulose derivative by weight, based on the total weight of the composition, with certain embodiments comprising about 1 to about 5% by weight of cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In some embodiments, the cellulose derivative is one or more of methylcellulose, HPC, HPMC, hydroxyethyl cellulose, and CMC. In some embodiments, the cellulose derivative is HPC. In some embodiments, the composition comprises from about 1 to about 3% HPC by weight, based on the total weight of the composition.

### Water

The water content of the composition, prior to use by a consumer of the composition, may vary according to the desired properties. Typically, the composition is less than about 60 percent by weight of water, and generally is from about 1 to about 60% by weight of water, for example, from about 5 to about 55, about 10 to about 50, about 20 to about 45, or about 25 to about 40 percent water by weight, including water amounts of at least about 5% by weight, at least about 10% by weight, at least about 15% by weight, and at least about 20% by weight. As described herein below, in some embodiments, the composition is enclosed in a pouch to form a pouched product. In some embodiments, the water content of the pouched product is in a range from about 40 weight percent to about 60 weight percent, based on the total weight of the pouched product (i.e., the composition and the pouch together). In some embodiments, the water content of the pouched product is in a range from about 15 weight percent to about 60 weight percent, based on the total weight of the pouched product (i.e., the composition and the pouch together). In some embodiments, the water content of the pouched product is in a range from about 15 weight percent to about 20 weight percent, based on the total weight of the pouched product (i.e., the composition and the pouch together). In some embodiments, the water content of the pouched product is in a range from about 30 weight percent to about 50 weight percent, based on the total weight of the pouched product (i.e., the composition and the pouch together), such as from about 32 to about 49 weight percent. In some embodiments, the water content of the pouched product is about 32, about 35, or about 48 weight percent, based on the total weight of the pouched product.

### Flavoring Agent

In some embodiments, the composition as described herein comprises a flavoring agent. As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the composition comprises a sensate which provides to the user of such composition a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA 4693), WS-116 (FEMA 4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-Hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N—(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one).

Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, menthol, menthyl esters, eucolyptol, WS-3, WS-23, WS-5, (1R,2S,5R)—N—(4-(cyanomethyl)phenyl)menthylcarboxamide (Evercool™ 180), (1R,2S,5R)—N—(2-(pyridin-2-yl)ethyl)menthylcarboxamide (Evercool™ 190), or a combination thereof.

In some embodiments, the composition does not comprise a flavoring agent, and comprises only a cooling agent to provide the desired user experience. In some embodiments, the cooling agent is WS-3.

In some embodiments, the composition comprises a modulator or sensate which provides to the user of such composition a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form. In some embodiments, a liquid flavorant is disposed (i.e., adsorbed or absorbed in or on) a porous particulate carrier, for example microcrystalline cellulose, which is then combined with the other composition ingredients.

The amount of flavoring agent utilized in the composition can vary, but is typically up to about 10% by weight, and certain embodiments are characterized by a flavoring agent content of at least about 0.1% by weight, such as about 0.5 to about 10%, about 1 to about 5%, or about 2 to about 4% weight, based on the total weight of the composition.

### Taste Modifiers

In order to improve the organoleptic properties of a composition as disclosed herein, the composition may include one or more taste modifying agents (“taste modifiers”) which may serve to mask, alter, block, or improve e.g., the flavor of a composition as described herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the composition comprises an ingredient having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component. Suitable taste modifiers include, but are not limited to, capsaicin, gamma-amino butyric acid (GABA), adenosine monophosphate (AMP), lactisole, or a combination thereof.

When present, a representative amount of taste modifier is about 0.01% by weight or more, about 0.1% by weight or more, or about 1.0% by weight or more, but will typically make up less than about 10% by weight of the total weight of the composition, (e.g., from about 0.01%, about 0.05%, about 0.1%, or about 0.5%, to about 1%, about 5%, or about 10% by weight of the total weight of the composition).

### Salts

In some embodiments, the composition may further comprise a salt (e.g., alkali metal salts), typically employed in an amount sufficient to provide desired sensory attributes to the composition. Non-limiting examples of suitable salts include sodium chloride, potassium chloride, ammonium chloride, flour salt, and the like.

When present, a representative amount of salt is about 0.5 percent by weight or more, about 1.0 percent by weight or more, or at about 1.5 percent by weight or more, but will typically make up about 10 percent or less of the total weight of the composition, or about 7.5 percent or less or about 5 percent or less (e.g., about 0.5 to about 5 percent by weight).

### Sweeteners

In order to improve the sensory properties of the composition according to the disclosure, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is sucralose, acesulfame K, or a combination thereof.

When present, a sweetener or combination of sweeteners may make up from about 0.01 to about 20% or more of the of the composition by weight, for example, from about 0.01 to about 0.1, from about 0.1 to about 1%, from about 1 to about 5%, from about 5 to about 10%, or from about 10 to about 20% by weight, based on the total weight of the composition. In some embodiments, a combination of sweeteners is present at a concentration of from about 0.01% to about 0.1% by weight of the composition, such as about 0.01, about 0.02, about 0.03, about 0.04, about 0.05, about 0.06, about 0.07, about 0.08, about 0.09, or about 0.1% by weight of the composition. In some embodiments, a combination of sweeteners is present at a concentration of from about 0.1% to about 0.5% by weight of the composition, such as about 0.1, about 0.2, about 0.3, about 0.4, or about 0.5% by weight of the composition. In some embodiments, a combination of sweeteners is present at a concentration of from about 1% to about 3% by weight of the composition.

### Composition Binding Agents

A binder (or combination of binders) may be employed in certain embodiments. Typical binders can be organic or inorganic, or a combination thereof. Representative binders include povidone, sodium alginate, starch-based binders, pectin, carrageenan, pullulan, zein, and the like, and combinations thereof. A binder may be employed in amounts sufficient to provide the desired physical attributes and physical integrity to the composition. The amount of binder utilized in the composition can vary, but is typically up to about 30 weight percent, and certain embodiments are characterized by a binder content of at least about 0.1% by weight, such as about 1 to about 30% by weight, or about 5 to about 10% by weight, based on the total weight of the composition.

Other suitable binders include a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the composition.

### Humectants

In certain embodiments, one or more humectants may be employed in the composition. Examples of humectants include, but are not limited to, polyols such as glycerin, propylene glycol, and the like. Where included, the humectant is typically provided in an amount sufficient to provide desired moisture attributes to the composition. Further, in some instances, the humectant may impart desirable flow characteristics to the composition for depositing in a mold.

When present, a humectant will typically make up about 5% or less of the weight of the composition (e.g., from about 0.5 to about 5% by weight). When present, a representative amount of humectant is about 0.1% to about 1% by weight, or about 1% to about 5% by weight, based on the total weight of the composition.

### Buffering Agents

In some embodiments, the composition of the present disclosure can comprise buffering agents. Examples of buffering agents that can be used include, but are not limited to, alkali metal buffers such as metal carbonates (e.g., potassium carbonate or sodium carbonate), metal bicarbonates such as sodium bicarbonate, alkali metal citrates, and the like. Non-limiting examples of suitable buffers include alkali or alkaline earth metal (e.g., sodium, potassium, calcium, magnesium) acetates, glycinates, phosphates, glycerophosphates, citrates, carbonates, hydrogen carbonates, borates, and mixtures thereof. In some embodiments, the buffer comprises acetate, phosphate or carbonate, such as an alkali metal acetate, phosphate, or carbonate. In some embodiments, the buffer comprises citrate. In some embodiments, the buffer comprises or is trisodium citrate. In some embodiments, the buffer is a mixture of trisodium citrate and citric acid. In some embodiments, the buffer is a solution of trisodium citrate and citric acid having a pH of about 6.3, such as a solution 0.5 molar in citrate.

When present, the buffering agent is typically present in an amount less than about 5 percent based on the weight of the composition, for example, from about 0.5% to about 5%, such as, e.g., from about 0.75% to about 4%, from about 0.75% to about 3%, or from about 1% to about 2% by weight, based on the total weight of the composition. As described herein above, such buffering agents may be added in a quantity sufficient to provide a specific pH value or range, or to maintain the pH of the composition within a desired range or at a desired value.

### Colorants

A colorant may be employed in amounts sufficient to provide the desired physical attributes to the composition. Natural or synthetic colorants, such as natural or synthetic dyes, food-grade colorants and pharmaceutical-grade colorants may be used. Examples of colorants include various dyes and pigments, such as caramel coloring and titanium dioxide. Natural colorants such as curcumin, beet juice extract, and spirulina, as well as a variety of synthetic pigments may also be used. The amount of colorant utilized in the composition can vary, but when present is typically up to about 3% by weight, such as from about 0.1%, about 0.5%, or about 1%, to about 3% by weight, based on the total weight of the composition.

### Oral Care Additives

In some embodiments, the composition comprises an oral care ingredient (or mixture of such ingredients). Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the composition. Other examples of ingredients that can be incorporated in desired effective amounts within the present composition can include those that are incorporated within the types of oral care compositions set forth in Takahashi et al., Oral Microbiology and Immunology, 19(1), 61-64 (2004); U.S. Pat. No. 6,083,527 to Thistle; and US Pat. Appl. Pub. Nos. 2006/0210488 to Jakubowski and 2006/02228308 to Cummins et al. Other example ingredients include those contained in formulations marketed as MALTISORB® by Roquette and DENTIZYME® by NatraRx. When present, a representative amount of oral care additive is at least about 1%, often at least about 3%, and frequently at least about 5% of the total dry weight of the composition. The amount of oral care additive within the composition will not typically exceed about 30%, often will not exceed about 25%, and frequently will not exceed about 20% of the total dry weight of the composition.

### Processing Aids

If necessary for downstream processing of the composition, such as granulation, mixing, or molding, a flow aid can also be added to the composition in order to enhance flowability of the composition. In some embodiments, the composition (e.g., melt and chew forms) may be surface treated with anti-stick agents, such as oils, silicones, and the like. Example flow aids include microcrystalline cellulose, silica, silicified microcrystalline cellulose, polyethylene glycol, stearic acid, calcium stearate, magnesium stearate, zinc stearate, sodium stearyl fumarate, carnauba wax, and combinations thereof. In some embodiments, the flow aid is sodium stearyl fumarate. In some embodiments, the composition comprises silicified microcrystalline cellulose as a flow aid and/or to enhance compressibility for tableting. Silicified microcrystalline cellulose is a combination of microcrystalline cellulose and colloidal silicon dioxide, commercially available as PROSOLV® SMCC from JRS Pharma.

When present, a representative amount of flow aid may make up at least about 0.5 percent or at least about 1 percent, of the total dry weight of the composition. Typically, the amount of flow aid within the composition will not exceed about 5 percent, and frequently will not exceed about 3 percent, of the total dry weight of the composition.

### Lipids

The composition can contain a lipid component, such as, for example, a fat, oil, or wax substance (or combination thereof). The lipid components used in the composition can be derived from animal or plant material and typically comprise mostly triglycerides along with lesser amounts of free fatty acids and mono- or di-glycerides. In certain embodiments, the lipid is a plant-derived fat material that is solid or semi-solid at room temperature (i.e., at about 25° C.) and which at least partially liquefies when subjected to the temperature of the oral cavity of the user. Such plant-derived fats are comprised primarily of saturated or unsaturated fatty acid chains (most of which are bound within triglyceride structures) having a carbon length of about 10 to about 26 carbon atoms, more typically about 14 to about 20 carbon atoms, and most often about 14 to about 18 carbon atoms. Example fats that can be used include palm oil, palm kernel oil, soybean oil, cottonseed oil, and mixtures thereof. According to some aspects, the lipid substance may be hydrogenated, partially hydrogenated, or non-hydrogenated. In some instances, the lipid substance may include a blend of lipid components. For example, the lipid substance may include a blend of palm oil and palm kernel oil.

A variety of methods exist for determining the melting point of lipids. However, many naturally occurring lipids are not chemically homogenous and, therefore, do not have a true single melting point, i.e., the temperature of transition from the solid to the liquid state. Thus, lipids are sometimes characterized by a melting range. One test for determining the melting point of lipids is the Mettler dropping point method (ASTM Standard D3954, 2010, “Standard Test Method for Dropping Point of Waxes,” ASTM International, West Conshohocken Pa., 2003, DOI 10.1520/D3954-94R10, www.astm.org.). Melting temperatures for lipids described herein, or products containing such lipids, refer to melting temperature points or ranges determined based on the Mettler dropping point method, unless otherwise specified.

The melting point of the lipid component may be chosen so that the resulting composition has a melting point in an appropriate range to provide the desired melting characteristics when the product is placed in the oral cavity of the user. In some embodiments, it may be desirable to use a lipid with a higher melting point so that the final product remains substantially solid in the oral cavity during use, meaning the user must chew the product in order to break the product into smaller pieces. In other embodiments, a lower melting point can be selected such that the final product readily melts in the mouth of the user during use. Typically, the melting point of the lipid component will be about 29° C. to about 49° C., often about 36° C. to about 45° C., and most often about 38° C. to about 41° C. In certain embodiments, the melting point of the product made using the lipid component will also have a melting point within the above ranges.

Lipid substances capable of use pursuant to the present disclosure are available from, for example, Loders Croklaan and AarhusKarlshamn USA Inc. Example lipid substances include 108-24-B from AarhusKarlshamn USA Inc. (a non-hydrogenated lauric coating fat containing a blend of palm kernel oil and palm oil); PARAMOUNT™ X from Loders Croklaan (a partially hydrogenated vegetable oil containing a blend of palm kernel oil, soybean oil, and cottonseed oil); CENTERNAL™ 625 from Loders Croklaan (a vegetable fat); PARAMOUNT™ C from Loders Croklaan (a lauric coating fat containing partially hydrogenated palm kernel oil with lecithin); KAOKOTE™ 102 from Loders Croklaan (a lauric coating fat containing partially hydrogenated palm kernel oil); SILKO™ 35-08 from AarhusKarlshamn USA Inc. (an interesterified hydrogenated lauric fat containing palm kernel oil); CEBES™ 27-70 from AarhusKarlshamn USA Inc. (a fractionated lauric cocoa butter substitute containing hydrogenated palm kernel oil); CISAO™ 82-53 from AarhusKarlshamn USA Inc. (a non-hydrogenated palm oil); CEBES™ 29-21 from AarhusKarlshamn USA Inc. (a fractionated cocoa butter substitute containing palm kernel oil and palm oil); CEBES™ 27-55 from AarhusKarlshamn USA Inc. (a lauric based fat containing partially hydrogenated palm kernel oil and hydrogenated soybean oil); 108-48-B from AarhusKarlshamn USA Inc. (a non-hydrogenated lauric fat which is a blend of palm kernel oil and palm oil); CEBES™ 29-07 from AarhusKarlshamn USA Inc. (a fractionated non-hydrogenated cocoa butter substitute); CEBES™ 21-20 from AarhusKarlshamn USA Inc. (a fractionated lauric cocoa butter substitute); CEBES™ 29-07 from AarhusKarlshamn USA Inc. (a fractionated lauric cocoa butter substitute); and CISAO™ 78-33 from AarhusKarlshamn USA Inc. (a non-hydrogenated structuring fat).

The relative amount of lipid substance within the composition may vary. In some embodiments, the amount of lipid substance within the composition is at least about 10 percent, at least about 20 percent, or at least about 30 percent, on a dry weight basis of the composition.

In some embodiments, the amount of lipid material is less than about 60 percent, less than about 50 percent, or less than about 40 weight percent, on a dry weight basis. Example lipid weight ranges include about 10 to about 60 dry weight percent, more typically about 20 to about 40 dry weight percent.

### Other Additives

Other additives can be included in the disclosed composition. For example, the composition can be processed, blended, formulated, combined and/or mixed with other materials or ingredients. The additives can be artificial or can be obtained or derived from herbal or biological sources. Examples of further types of additives include thickening or gelling agents (e.g., fish gelatin), emulsifiers, preservatives, antioxidants, disintegration aids, or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference.

In some embodiments, the composition comprises one or more antioxidants, one or more preservatives, one or more antimicrobial agents, or combinations thereof. Suitable antioxidants and preservatives include, but are not limited to, ascorbic acid (Vitamin C), sodium ascorbate, ascorbyl palmitate, butylated hydroxytoluene (BHT), butylated hydroxyanisole (BHA), tertiary butylhydroquinone (TBHQ), methyl paraben, propyl paraben, sodium propionate, potassium sorbate, sodium benzoate, propyl gallate, monosterol citrate, Vitamin E, derivatives of vitamin E, tocopherols, and combinations thereof.

Typical inclusion ranges for such additional additives can vary depending on the nature and function of the additive and the intended effect on the final composition, with an example range of up to about 10% by weight, based on total weight of the composition (e.g., about 0.1 to about 5% by weight).

The aforementioned additives can be employed together (e.g., as additive formulations) or separately (e.g., individual additive components can be added at different stages involved in the preparation of the final mixture). Furthermore, the aforementioned types of additives may be encapsulated as provided in the final product or composition. Example encapsulated additives are described, for example, in WO2010/132444 to Atchley, which has been previously incorporated by reference herein.

### Configured for Oral Use

Provided herein are compositions configured for oral use. The term “configured for oral use” as used herein means that the composition or product including such composition is provided in a form such that during use, saliva in the mouth of the user causes one or more of the releasable components of the composition (e.g., flavoring agents and/or substituted 3-(1-methylpyrrolidin-2-yl)pyridine) to pass into the mouth of the user. In some embodiments, the composition or product is adapted to deliver components to a user through mucous membranes in the user's mouth and, in some instances, said component is a flavoring agent and/or a substituted 3-(1-methylpyrrolidin-2-yl)pyridine as described herein that can be absorbed through the mucous membranes in the mouth when the composition or product is used.

In some embodiments, the composition or product including such composition could have the potential to be used as an aid in smoking cessation, as a nicotine replacement therapy, and/or the treatment of nicotine addiction.

Products configured for oral use as described herein may take various forms, including gels, pastilles, gums, lozenges, powders, and pouches. Certain products of the disclosure are in the form of solids. Certain products can exhibit, for example, one or more of the following characteristics: crispy, granular, chewy, syrupy, pasty, fluffy, smooth, and/or creamy. In some embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

Any of the product formats disclosed herein may also be made into effervescing products by incorporating an effervescent material capable of causing effervescence in the oral cavity into the composition. Suitable effervescent materials may comprise an acid component and a base component, wherein the base component is a carbonate material, a bicarbonate material, or a mixture thereof, and the acid component is a tricarboxylic acid, a dicarboxylic acid, a monocarboxylic acid, or a combination thereof. Alternatively, suitable effervescent materials include those comprising a sugar material containing an entrapped gaseous component, such that release of the entrapped gaseous component occurs upon dissolution of the sugar material in the oral cavity.

The compositions as disclosed herein can be formed into a variety of shapes, including pills, tablets, spheres, strips, films, sheets, coins, cubes, beads, ovoids, obloids, cylinders, bean-shaped, sticks, or rods. Cross-sectional shapes of the composition can vary, and example cross-sectional shapes include circles, squares, ovals, rectangles, and the like.

The compositions of the present disclosure may be at least partially dissolvable. As used herein, the terms “dissolve,” “dissolving,” and “dissolvable” refer to compositions having aqueous-soluble components that interact with moisture in the oral cavity and enter into solution, thereby causing gradual consumption of the composition. In some embodiments, the dissolvable composition is capable of lasting in the user's mouth for a given period of time until it completely dissolves. Dissolution rates can vary over a wide range, from about 1 minute or less to about 60 minutes. For example, fast release compositions typically dissolve and/or release the substituted 3-(1-methylpyrrolidin-2-yl)pyridine in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the composition.

In some embodiments, the composition can be meltable as discussed, for example, in US Patent App. Pub. No. 2012/0037175 to Cantrell et al., incorporated by reference herein in its entirety. As used herein, “melt,” “melting,” and “meltable” refer to the ability of the composition to change from a solid state to a liquid state. That is, melting occurs when a substance (e.g., a composition as disclosed herein) changes from solid to liquid, usually by the application of heat. The application of heat in regard to a composition as disclosed herein is provided by the internal temperature of a user's mouth. Thus, the term “meltable” refers to a composition that is capable of liquefying in the mouth of the user as the composition changes phase from solid to liquid, and is intended to distinguish compositions that merely disintegrate in the oral cavity through loss of cohesiveness within the composition that merely dissolve in the oral cavity as aqueous-soluble components of the composition interact with moisture. Generally, meltable compositions comprise a lipid as described herein above. In some embodiments, the composition in meltable form comprises a lipid in an amount of from about 35 to about 50% by weight, based on the total weight of the composition, and a sugar alcohol in an amount of from about 35 to about 55% by weight, based on the total weight of the composition. In some embodiments, the sugar alcohol is isomalt, erythritol, sorbitol, arabitol, ribitol, maltitol, dulcitol, iditol, mannitol, xylitol, lactitol, or a combination thereof. In some embodiments, the sugar alcohol is isomalt.

In some embodiments, the composition is in the form of a compressed or molded pellet or tablet. Such pellets or tablets generally comprise at least one filler, at least one sugar alcohol, and may further include flavorants, binders, salts, sweeteners, buffering agents, colorants, humectants, oral care additives, preservatives, disintegration aids, flow aids, compressibility aids, or combinations thereof, each as described herein above. Generally, the composition in tablet form comprises a glucose-polysaccharide blend and a sugar alcohol. In some embodiments, the glucose-polysaccharide blend is present in an amount of from about 35 to about 50% by weight, based on the total weight of the composition; and the sugar alcohol is present in an amount of from about 30 to about 45% by weight, based on the total weight of the composition. Example pellet weights range from about 250 mg to about 1500 mg, such as about 250 mg to about 700 mg, or from about 700 mg to about 1500 mg. The pellet can have any of a variety of shapes, including traditional pill or tablet shapes.

In some embodiments, the composition can be chewable, meaning the composition has a mild resilience or “bounce” upon chewing, and possesses a desirable degree of malleability. A composition in chewable form may be entirely dissolving or may be in the form of a non-dissolving gum in which only certain components (e.g., active ingredients, flavor, sweetener) dissolve, leaving behind a non-dissolving matrix. Chewable embodiments generally include a binder, such as a natural gum or pectin. In some embodiments, the composition in chewable form comprises pectin and an organic acid, along with one or more sugar alcohols in an amount by weight of at least 50%, based on the total weight of the composition. Generally, the pectin is present in an amount of from about 1 to about 3% by weight, based on the total weight of the composition.

In some embodiments, the composition is in the form of a gum, such as a chewing gum. Chewing gum compositions generally comprise a gum base and one or more fillers, and may further comprise emulsifiers, flavoring agents, salts, sweeteners, humectants, binders, buffering agents, colorants, humectants, oral care additives, preservatives, or combinations thereof, each as described herein above. By the term “gum base” is meant the generally water-insoluble and hydrophobic ingredients that provide the desired texture to the gum. The chewing gum may comprise the gum base in an amount from about 5 to about 95 percent by weight. Generally, the gum base comprises an elastomer. Elastomers provide the rubbery, cohesive nature to the gum base and the overall chewing gum. Elastomers suitable for use in the gum base of the present disclosure may include natural, synthetic, or a combination thereof. Non-limiting examples of suitable natural elastomers include latex, chicle, crown gum, nispero, rosidinha, jelutong, perillo, niger gutta, tunu, balata, guttapercha, lechi caspi, sorva, gutta kay, massaranduba balata, massaranduba chocolate, nispero, rosindinha, gutta hang kang, chiquibul, pendare, leche de vaca, tunu, chilte, and mixtures thereof. Non-limiting examples of suitable synthetic elastomers include styrene-butadiene copolymers (SBR), polyisobutylene, isobutylene-isoprene copolymers, polyethylene, polyvinyl acetate, biodegradable polymers, and mixtures thereof. Further examples of synthetic elastomers include, but are not limited to, synthetic elastomers listed in Food and Drug Administration, CFR, Title 21, Section 172,615, the Masticatory Substances, Synthetic.

Suitable bulking agents include sweeteners such as sugars, sugar alcohols, and combinations thereof.

Gum bases, and particularly synthetic elastomer-based gum bases, may further comprise one or more of fillers, plasticizers, softeners, emulsifiers, waxes, anti-tacking agents, and further miscellaneous components such as antioxidants, colorants, flavoring agents, buffers, and the like. Suitable fillers include, but are not limited to, magnesium carbonate, calcium carbonate, ground limestone, silicates, such as magnesium and aluminum silicate, clay, alumina, talc, titanium oxide, mono-, di- and tri-calcium phosphate, cellulose polymers, and combinations thereof. In some embodiments, the filler is calcium carbonate, talc, or a combination thereof. In some embodiments, the filler is talc. Suitable plasticizers include natural resins, synthetic resins, waxes, and combinations thereof. Suitable emulsifiers and/or softeners include, but are not limited to, tallow, hydrogenated tallow, hydrogenated and partially hydrogenated vegetable oils, cocoa butter, glycerol monostearate, glycerol acetate, glycerol diacetate (diacetin) glycerol triacetate (triacetin), lecithin, mono-, di- and triglycerides, acetylated monoglycerides, fatty acids (e.g., stearic, palmitic, oleic and linoleic acids), sorbitan fatty acid esters, lanolin, and combinations thereof.

In some embodiments, the composition may be in the form of a dissolvable and lightly chewable pastille. As used herein, the term “pastille” refers to a dissolvable oral product made by solidifying a liquid or gel composition, such as a composition that includes a gelling or binding agent, so that the final product is a hardened solid gel. A pastille product may alternatively be referred to as a soft lozenge. In some embodiments, the pastille products of the disclosure are characterized by sufficient cohesiveness to withstand light chewing action in the oral cavity without rapidly disintegrating. The pastille products of the disclosure typically do not exhibit a highly deformable chewing quality as found in conventional chewing gum. See, for example, the pastille formulations, pastille configurations, pastille characteristics and techniques for formulating or manufacturing pastilles set forth in U.S. Pat. No. 9,204,667 to Cantrell et al.; U.S. Pat. No. 9,775,376 to Cantrell et al.; U.S. Pat. No. 10,357,054 to Marshall et al.; which are incorporated herein by reference.

In some embodiments, the products disclosed herein may be in the form of a dissolvable lozenge product configured for oral use. Lozenge products are generally described as “hard” and are distinguished in this manner from soft lozenges (i.e., pastilles). Example lozenge-type products have the form of a lozenge, tablet, microtab, or other tablet-type product. See, for example, the types of nicotine-containing lozenges, lozenge formulations, lozenge formats and configurations, lozenge characteristics and techniques for formulating or manufacturing lozenges set forth in U.S. Pat. No. 4,967,773 to Shaw; U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al.; U.S. Pat. No. 6,248,760 to Wilhelmsen; and U.S. Pat. No. 7,374,779; and U.S. Pat. Pub. Nos. 2001/0016593 to Wilhelmsen; 2004/0101543 to Liu et al.; 2006/0120974 to Mcneight; 2008/0020050 to Chau et al.; 2009/0081291 to Gin et al.; and 2010/0004294 to Axelsson et al., each of which is incorporated herein by reference.

In some embodiments, the products disclosed herein may be in the form of a transparent or translucent lozenge. Transparency/translucency can be determined by any means commonly used in the art; however, it is commonly measured by spectrophotometric light transmission over a range of wavelengths (e.g., from about 400-700 nm). Transmission measurements for such transparent or translucent lozenge products are typically higher than those of traditional (i.e., non-translucent) products. Translucency can also be confirmed by visual inspection by simply holding the product up to a light source and determining if light travels through the product in a diffuse manner. Translucent lozenge products generally comprise as the lozenge base a sugar substitute in an amount of at least about 80% by weight, and a sugar alcohol syrup. The amount and type of the sugar substitute can vary. In some embodiments, the sugar substitute is a non-hygroscopic sugar alcohol capable of forming a glassy matrix. For example, in some embodiments, the sugar substitute is isomalt. In some embodiments, the sugar substitute is present in an amount of at least about 85% by weight or at least about 90% by weight. In some embodiments, the sugar alcohol syrup is maltitol syrup. Although sugar alcohol syrups may be preferred, sugar syrups can, in some embodiments, be used in place of or in combination with the sugar alcohol syrup. For example, in some embodiments, corn syrup, golden syrup, and/or molasses can be used.

In some embodiments, the products disclosed herein may be in the form of a film, such as an edible or dissolving film. Such films generally comprise a mixture of one or more fillers, emulsifiers, gelling agents, and binders to provide the desired film format, along with additional components such as buffers, humectants, salts, sweeteners, and the like. In some embodiments, the composition can be chewable, meaning the composition has a mild resilience or “bounce” upon chewing, and possesses a desirable degree of malleability. A composition in chewable form may be entirely dissolving or may be in the form of a non-dissolving gum in which only certain components (e.g., substituted 3-(1-methylpyrrolidin-2-yl)pyridine, flavor, sweetener) dissolve, leaving behind a non-dissolving matrix.

In some embodiments, the composition can be meltable as discussed, for example, in US Patent App. Pub. No. 2012/0037175 to Cantrell et al., incorporated by reference herein in its entirety. As used herein, “melt,” “melting,” and “meltable” refer to the ability of the composition to change from a solid state to a liquid state. That is, melting occurs when a substance (e.g., a composition as disclosed herein) changes from solid to liquid, usually by the application of heat. The application of heat in regard to a composition as disclosed herein is provided by the internal temperature of a user's mouth. Thus, the term “meltable” refers to a composition that is capable of liquefying in the mouth of the user as the composition changes phase from solid to liquid and is intended to distinguish compositions that merely disintegrate in the oral cavity through loss of cohesiveness within the composition that merely dissolve in the oral cavity as aqueous-soluble components of the composition interact with moisture.

Certain types of products disclosed herein (e.g., lozenges, tablets, and the like) also can have outer coatings composed of ingredients capable of providing acceptable outer coatings (e.g., an outer coating can be composed of ingredients such as carnauba wax, and pharmaceutically acceptable forms of shellacs, glazing compositions and surface polish agents). Application of a coating can be accomplished using techniques such as airless spraying, fluidized bed coating, use of a coating pan, or the like. Materials for use as a coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Compositions can be co-extruded, laminated or formed so as to form products having sandwich-type forms, and hence the location of ingredients (e.g., substituted 3-(1-methylpyrrolidin-2-yl)pyridine) can be controlled in order to provide the desired features such as performance, behavior, interaction or non-interaction with other ingredients, storage stability, and the like. In addition, mixtures of component ingredients can be formulated and manufactured into core/shell types of configurations (e.g., products that have an inner region and at least one additional overlayer), with the various regions of such products having differing overall compositions or properties. Thus, for example, the substituted 3-(1-methylpyrrolidin-2-yl)pyridine can have a relatively high concentration towards the inner region of the product, or a relatively high concentration towards the outer region of the product. Other configurations are contemplated herein, such as multi-layered compositions comprising two or more formulations having different organoleptic properties. Non-limiting embodiments of multi-layer compositions include but are not limited to lozenge/meltable, lozenge/chewable, lozenge/hard coating, meltable/lozenge, meltable/hard coating, molded/meltable, molded/lozenge, lozenge/meltable/hard coating, and molded/lozenge/meltable. In some embodiments, the composition is a core formulation surrounded by one or more continuous layers or a core formulation coated by one or more discontinuous (e.g., partial) layers so as to form a layered or side-by-side configuration of the two or more formulations. The number of layers can vary; for example, the multi-layered composition can in some embodiments comprise between two and ten layers, such as between two and five layers. Such layered or core-shell embodiments can be formed into a desired format (e.g., tablet, lozenge, pastille, gum, film, etc.) by pouring or otherwise introducing a first mixture into a mold, injection molding a first mixture, or other using other suitable means for providing a formulation. In some embodiments, a second mixture can be applied by spray coating, dip coating, or by forming the second mixture into a sheet that is applied to the first mixture as a sandwiched coating. Spray coating or dip coating can be conducted at a temperature such that the first mixture is maintained in substantially intact form. In some embodiments, a third mixture is applied as a coating on the second mixture. The various mixtures utilized for the layers or core and shell may vary in terms of physical properties (hardness, meltability, chewiness, dissolvability, and the like), flavor, presence or absence of substituted 3-(1-methylpyrrolidin-2-yl)pyridine, concentration of substituted 3-(1-methylpyrrolidin-2-yl)pyridine, or combinations thereof.

In some embodiments, the composition of the present disclosure is disposed within a moisture-permeable container (e.g., a water-permeable pouch) to form a pouched oral product. Such pouched products are typically used by placing one pouch containing the composition in the mouth of a human subject/user. Generally, the pouch is placed somewhere in the oral cavity of the user, for example between lips and gums, in the same way as moist snuff products are generally used. Exposure to saliva then causes some of the components of the composition therein (e.g., flavoring agents and/or nicotine) to pass through e.g., the water-permeable pouch and provide the user with flavor and satisfaction, and the user is not required to spit out any portion of the composition. After about 10 minutes to about 60 minutes, typically about 15 minutes to about 45 minutes of use/enjoyment, substantial amounts of the composition have been absorbed by the human subject, and the pouch may be removed from the mouth of the human subject for disposal. The pouch generally is not chewed and the pouch is not swallowed, nor is the pouch intended for ingestion. In other words, certain components present in the composition (e.g., flavoring agents and/or substituted 3-(1-methylpyrrolidin-2-yl)pyridine) are consumed during use of the pouched product, but the pouch itself is not ingested (unless the pouch is a dissolving pouch as described further herein below).

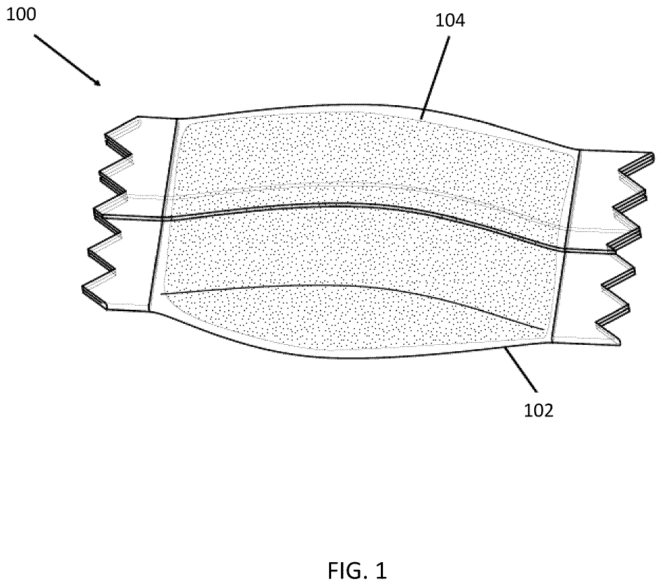

Accordingly, in some embodiments, the composition as disclosed herein, and any other components noted above, are combined within a moisture-permeable packet or pouch that acts as a container for use of the composition to provide a pouched product configured for oral use. Some embodiments of the disclosure may be described with reference to FIG. 1, which provides an illustration of an example pouched product. Referring to FIG. 1, there is shown an embodiment of a pouched product 100. The pouched product 100 includes a moisture-permeable container in the form of a pouch 102 housing a material 104 comprising the oral composition described herein.

An example pouch 102 can be constructed of a fleece material, which is a fibrous material that can be formed from various types of fibers (e.g., conventional cellulosic fibers (e.g., such as viscose fibers, regenerated cellulose fibers, cellulose fibers, and wood pulps), cotton fibers, wool fibers, other natural fibers, polymer/synthetic-type fibers, and combinations thereof) capable of being formed into a fabric. For example, fleece materials may be provided in the form of a woven or nonwoven fabric. Suitable types of fleece materials, for example, are described in U.S. Pat. No. 8,931,493 to Sebastian et al.; US Patent App. Pub. No. 2016/0000140 to Sebastian et al.; and US Patent App. Pub. No. 2016/0073689 to Sebastian et al., which are all incorporated herein by reference.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and composition each may undergo complete dispersion within the mouth of the user during normal conditions of use, and hence the pouch and composition both may be consumed by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). If desired, flavoring ingredients, disintegration aids, and other desired components, may be incorporated within, or applied to, the pouch material.

The amount of material contained within each product unit, for example, a pouch, may vary. In some embodiments, the dry weight of the composition within each pouch is at least about 50 mg, for example, from about 50 mg to about 1.3 grams, from about 100 to 800 about mg, from about 200 to about 750 mg, or from about 700 to about 1330 mg. In some embodiments, the product is a small pouch, and the dry weight of the composition within each pouch may be from about 100 to about 300 mg. In some embodiments, the product is a large pouch, and the dry weight of the material within each pouch may be from about 700 mg to about 1.3 grams. If desired, other components can be contained within each pouch. For example, at least one flavored strip, piece or sheet of flavored water dispersible or water-soluble material (e.g., a breath-freshening edible film type of material) may be disposed within each pouch. Such strips or sheets may be folded or crumpled in order to be readily incorporated within the pouch. See, for example, the types of materials and technologies set forth in U.S. Pat. No. 6,887,307 to Scott et al. and U.S. Pat. No. 6,923,981 to Leung et al.; and The EFSA Journal (2004) 85, 1-32; which are incorporated herein by reference.

In some embodiments, the pouch may include one or more capsules, such as microcapsules. In some embodiments, the capsule(s) include at least a portion of the substituted 3-(1-methylpyrrolidin-2-yl)pyridine. The capsule can comprise a capsule wall that is formed of a material that is configured to allow for dispersal of the contents under desired conditions. For example, the capsule wall may comprise a material that is configured to dissolve or otherwise degrade under mouth conditions. As another non-limiting example, the capsule wall may comprise a material that is configured to be broken or otherwise degraded by shear forces (e.g., chewing). While suitable encapsulated materials may be described herein in relation to microcapsules, it is understood that such terminology is not intended to be viewed as limiting of the capsule sizes.

The crush strength of suitable microcapsules can be sufficient to allow for normal handling and storage without significant degree of premature or undesirable breakage, Providing capsules that possess both suitable integrity during storage and the ability to rupture or otherwise break down at the time of use can be determined by experimentation, depending upon factors such as capsule size and type, and is a matter of design choice. See, for example, U.S. Pat. Pub. No, 2007/0068540 to Thomas et al., which is incorporated herein by reference,

In some embodiments, the capsule may include an outer shell incorporating a material such as wax, gelatin, cyclodextrin, or alginate, and an inner payload. In some embodiments, the inner payload (e.g., a substituted 3-(1-methylpyrrolidin-2-yl)pyridine) is in particulate form or is present as an aqueous or non-aqueous solution or dispersion within water or an organic liquid such as an alcohol, oil, glycerin or combination thereof), Thus, for example, a plurality of such capsules may be incorporated within the pouch along with the composition to be retained therein, and during use of the product, a crushing or other destruction of the capsules allows the capsules to release the payload contained therein.

The capsules may be uniform or varied in size, weight. and shape. A representative capsule can be generally spherical in shape. However, suitable capsules may have other types of shapes, such as generally rectilinear, oblong, elliptical, or oval shapes, In some embodiments, microcapsules may have diameters of less than about 100 microns, such as microcapsules having diameters in the range of about 1 to about 40 microns, or about 1 micron to about 20 microns.

In some embodiments, larger capsules may be utilized. For example, a capsule utilized in the pouch product may have a size of about 0.5 mm to about 5 mm or about 0.6 mm to about 3 mm in diameter,

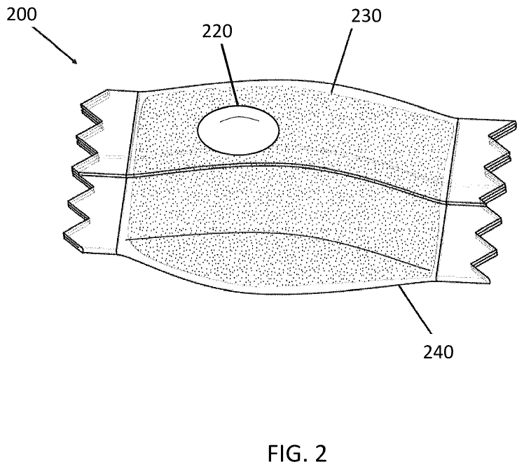

The number of capsules incorporated into the pouched product can vary, depending upon factors such as the size of the capsules, the character or nature of the payload, the desired attributes of the composition within the pouch, and the like, In some embodiments, only a single capsule is included. The single capsule can be relatively large in size. For example, as seen in the pouched product 200 of FIG. 2, a capsule 220 having the payload (e.g., a substituted 3-(1-methylpyrrolidin-2-yl)pyridine) (not visible) provided therein may be positioned within the water permeable fabric 240 along with the composition 230 within. The single capsule 220 can thus be relatively large in size.

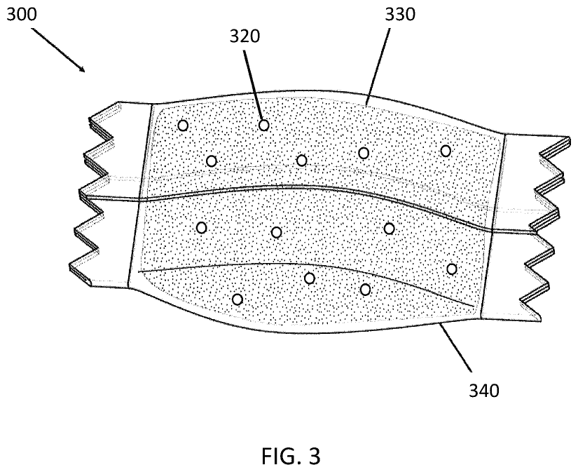

In some embodiments, a plurality of capsules may be included, and may be positioned within the pouch fabric along with the composition. The plurality of capsules may be relatively small in size. In particular, microcapsules may be used. For example, as seen in the pouched product 300 of FIG. 3, a plurality of capsules 320 each having the same or a different payload (e.g., a substituted 3-(1-methylpyrrolidin-2-yl)pyridine) (not visible) provided therein may be positioned within the water permeable fabric 340 along with the composition 330 within.

The plurality of capsules 320 can thus be relatively small in size. The number of capsules incorporated within pouched product, for example, can exceed about 5, can exceed about 10, can exceed about 20, can exceed about 40, and can even exceed about 100. In certain embodiments, the number of capsules/microcapsules can be greater than about 500, and even greater than about 1,000.

The total weight of the capsules contained within the product may vary, but is typically greater than about 10 mg, often greater than about 20 mg, and can be greater than about 30 mg. The total weight of the capsules is typically less than about 200 mg, often less than about 100 mg, and can be less than about 50 mg.

The capsules can be formed using any encapsulating technology known in the art. For example, microcapsules can be formed using any of various chemical encapsulation techniques such as solvent evaporation, solvent extraction, organic phase separation, interfacial polymerization, simple and complex coacervation, in-situ polymerization, liposome encapsulation, and nanoencapsulation, Alternatively, physical methods of encapsulation could be used, such as spray coating, pan coating, fluid bed coating, annular jet coating, spinning disk atomization, spray cooling, spray drying, spray chilling, stationary nozzle coextrusion, centrifugal head coextrusion, or submerged nozzle coextrusion.

Regardless of the encapsulation methodology employed, the outer wall or shell material and solvents used to form the capsules can vary. Classes of materials that are typically used as wall or shell materials include proteins, polysaccharides, starches, waxes, fats, natural and synthetic polymers, and resins, Exemplary materials for use in the microencapsulation process used to form the microcapsules include gelatin, acacia (gum arabic), polyvinyl acetate, potassium alginate, carob bean gum, potassium citrate, carrageenan, potassium polymetaphosphate, citric acid, potassium tripolyphosphate, dextrin, polyvinyl alcohol, povidone, dimethylpolysiloxane, dimethyl silicone, refined paraffin wax, ethylcellulose, bleached shellac, modified food starch, sodium alginate, guar gum, sodium carboxymethylcellulose, hydroxypropyl cellulose, sodium citrate, hydroxypropylmethyicellulose, sodium ferrocyanide, sodium polyphosphates, locust bean gum, methylcellulose, sodium trimetaphosphate, methyl ethyl cellulose, sodium tripolyphosphate, microcrystalline wax, tannic acid, petroleum wax, terpene resin, tragacanth, polyethylene, xanthan gum, and polyethylene glycol.

Microcapsules are commercially available, and exemplary types of microcapsule technologies are of the type set forth in Gutcho, Microcapsules and Microencapsulation Techniques (1976); Gutcho, Microcapsules and Other Capsules Advances Since 1975 (1979); Kondo, Microcapsule Processing and Technology (1979); Iwamoto et al., AAPS Pharm. Sci. Tech. 2002 3(3): article 25; U.S. Pat. No. 3,550,598 to McGlumphy: U.S. Pat. No. 4,889,144 to Tateno et al.; U.S. Pat. No. 5,004,595 to Cherukuri et al.; U.S. Pat. No. 5,690,990 to Bonner; U.S. Pat. No. 5,759,599 to Wampler et al.; U.S. Pat. No. 6,039,901 to Soper et at.; U.S. Pat. No. 6,045,835 to Soper et al.: U.S. Pat. No. 6,056,992 to Lew; U.S. Pat. No. 6,106,875 to Soper et al.; U.S. Pat. No. 6,117,455 to Takada et al.; U.S. Pat. No. 6,325,859 to DeRoos et al.; U.S. Pat. No. 6,482,433 to DeRoos et al.; U.S. Pat. No. 6,612,429 to Dennen; and U.S. Pat. No. 6,929,814 to Bouwmeesters et al.: U.S. Pat. Appl. Pub. Nos. 2006/0174901 to Karles et al, and 2007/0095357 to Besso et al.; and PCT WO2007/037962 to Holton et a.; each of which is incorporated herein by reference. Suitable types of microcapsules are available from sources such as Microtek Laboratories of Dayton, Ohio. Exemplary types of commercially available microencapsulating techniques include those marketed under the trade names ULTRASEAL™ and PERMASEAL™, available from Givaudan headquartered in Vernier, Switzerland.

Representative types of capsules are of the type commercially available as “Momints” by Yosha! Enterprises, Inc. and “Ice Breakers Liquid Ice” from The Hershey Company. Representative types of capsules also have been incorporated in chewing guru, such as the type of gum marketed under the tradename “Cinnaburst” by Cadbury Adams USA. Representative types of capsules and components thereof also are set forth in U.S. Pat. No. 3,339,558 to Waterbury; U.S. Pat. No. 3,390,686 to Irby, Jr. et al.; U.S. Pat. No. 3,685,521 to Dock; U.S. Pat. No. 3,916,914 to Brooks et al.; 1U.S. Pat. No. 4,889,144 to Tateno et al, U.S. Pat. No. 6,631,722 to MacAdam et al.; and U.S. Pat. No. 7,115,085 to Deal; US Pat., Pub. Nos. 2004/0261807 to Dube et al.; 2006/0272663 to Dube et al.; 2006/01330961 to Luan et al.; 2006/0144412 to Mishra et al; 2007/0012327 to Karles et al; and 2007/0068540 to Thomas et al.; PCT WO 03/009711 to Kim; PCT WO2006/136197 to Hartmann et al; PCT WO 2006/136199 to Mane et al., PCT WO 2007/010407; and PCT WO 2007/060543, as well as within filtered cigarettes that have been marketed under the tradename “Camel Lights with Menthol Boost” by R. J. Reynolds Tobacco Company, which are incorporated herein by reference. See also, the types of capsules and components thereof set forth in U.S. Pat. No. 5,223,185 to Takei et al.: U.S. Pat. No. 5,387,093 to Takei: U.S. Pat. No. 5,882,680 to Suzuki et al.; U.S. Pat. No. 6,719,933 to Nakamura et al. and U.S. Pat. No. 6,949,256 to Fonkwe et at; and U.S. Pat. App. Pub. Nos. 2004/0224020 to Schoenhard; 2005/0123601 to Mane et al.; 2005/0196437 to Bednarz et al.; and 2005/0249676 to Scott et al.; which are incorporated herein by reference. The capsules may be colored, provided with smooth or rough surfaces, have rigid or pliant shells, have brittle or durable shells, or other desired features or characters,

In pouched embodiments comprising a fleece material as the pouch, the amount of substituted 3-(1-methylpyrrolidin-2-yl)pyridine (e.g., 2-methyl-5-(1-methylpyrrolidin-2-yl)pyridine) present within the fleece material may vary. For example, some of the substituted 3-(1-methylpyrrolidin-2-yl)pyridine (e.g., 2-methyl-5-(1-methylpyrrolidin-2-yl)pyridine) may migrate from the composition into the fleece during storage. In some embodiments, a portion of the substituted 3-(1-methylpyrrolidin-2-yl)pyridine (e.g., 2-methyl-5-(1-methylpyrrolidin-2-yl)pyridine) is added to the fleece material during manufacture. Accordingly, the amount of substituted 3-(1-methylpyrrolidin-2-yl)pyridine (e.g., 2-methyl-5-(1-methylpyrrolidin-2-yl)pyridine) present within the fleece (i.e., adsorbed or absorbed) may range from 0% up to 75% or more by weight, based on the total amount of substituted 3-(1-methylpyrrolidin-2-yl)pyridine (e.g., 2-methyl-5-(1-methylpyrrolidin-2-yl)pyridine) present in the product as a whole. In some embodiments, the fleece includes less than 10%, such as less than 5%, less than 1%, less than 0.1%, or even 0% of the total substituted 3-(1-methylpyrrolidin-2-yl)pyridine (e.g., 2-methyl-5-(1-methylpyrrolidin-2-yl)pyridine) present in the product by weight. In some embodiments, at least a portion of the total amount of substituted 3-(1-methylpyrrolidin-2-yl)pyridine (e.g., 2-methyl-5-(1-methylpyrrolidin-2-yl)pyridine) in the product by weight is present in the fleece (i.e., adsorbed or absorbed), such as at least 1% and up to 75% or more. In some embodiments, the fleece comprises from about 1%, about 5%, about 10%, about 20%, about 30%, about 40%, about 50%, about 60%, about 70%, about 80%, or about 90% of the total amount of substituted 3-(1-methylpyrrolidin-2-yl)pyridine (e.g., 2-methyl-5-(1-methylpyrrolidin-2-yl)pyridine) present by weight in the product.

In some embodiments, the composition is in the form of a snuff or “dip”, which may be loose or may be enclosed in a fleece material to form a pouched product. The composition in the form of a snuff comprises, in addition to a substituted 3-(1-methylpyrrolidin-2-yl)pyridine as described herein a non-tobacco botanical material. The composition may further comprise components such as water, salts, humectants, buffers, sweeteners, binders, lipids, and flavorants, each as described herein.

Suitable non-tobacco materials are generally fibrous plant derived materials such as corn silk, red clover, wheat fiber, oat fiber, sugarcane fiber, beet pulp, barley fiber, rye fiber, buckwheat fiber, sugar beet fiber, bran fiber, bamboo fiber, wood pulp fiber, cotton fiber, citrus fiber, grass fiber, willow fiber, poplar fiber, cocoa fiber, and combinations thereof. In some embodiments, the non-tobacco plant fiber material is bamboo fiber or sugarcane fiber. For the avoidance of doubt, non-tobacco plant fiber materials do not encompass microcrystalline cellulose. Suitable non-tobacco plant fiber materials include but are not limited to commercially available products such as products sold under the brand name ALBAFIBER (Azelis), products sold under the brand name JELUCEL (Jelu-Werk; J. Ehrler GmbH & Co. KG), products sold by Natural Fiber Solutions (NFS), products sold under the UNICELL brand name (InterFiber), products sold by Domsjö Fiber AB, and the like.

In some embodiments, the snuff composition further comprises a sensate (such as N-ethyl-p-menthane-3-carboxamide [WS-3], capsaicin, alpha-sanshool, and the like), natural and/or artificial sweeteners (e.g., allulose, sucralose, saccharin, acesulfame K, glycyrrhizin (licorice extract), stevia, mogroside, xylitol, combinations, and the like), a binder (such as guar gum, xanthan gum, carboxymethylcellulose, hydroxypropyl cellulose, and the like), coloring agents (dyes, caramel color, and the like), and glycerides (such as MCT oil).

In some embodiments, products of the disclosure, when placed in the oral cavity of a user, exhibit favorable pharmacokinetic properties (e.g., time to maximum concentration (Tₘₐₓ), maximal concentration (Cₘₐₓ), exposure (AUC), or half-life (T_{1/2})). For example, in some embodiments, pouched products of the disclosure comprising 2-methyl-5-(1-methylpyrrolidin-2-yl)pyridine, when placed in the oral cavity of a user, exhibit pharmacokinetic properties consistent with or superior to those of a comparable pouch containing nicotine.

In some embodiments, a pouched product of the disclosure comprising 2-methyl-5-(1-methylpyrrolidin-2-yl)pyridine when placed in the oral cavity of a user, exhibits a T_{1/2} of 2-methyl-5-(1-methylpyrrolidin-2-yl)pyridine which is similar to or longer than the T_{1/2} of a comparable pouched product containing nicotine. Without wishing to be bound by theory, it is believed that a longer T_{1/2} may be due to decreased metabolism of the 2-methyl-5-(1-methylpyrrolidin-2-yl)pyridine relative to nicotine.

In some embodiments, a pouched product of the disclosure comprising 2-methyl-5-(1-methylpyrrolidin-2-yl)pyridine when placed in the oral cavity of a user, exhibits a Tmax of 2-methyl-5-(1-methylpyrrolidin-2-yl)pyridine which is similar to or shorter than the Tₘₐₓ of a comparable pouched product containing nicotine. Without wishing to be bound by theory, it is believed that a longer Tₘₐₓ may be due to increased Log P relative to nicotine.

In some embodiments, a pouched product of the disclosure comprising 2-methyl-5-(1-methylpyrrolidin-2-yl)pyridine when placed in the oral cavity of a user, exhibits a Cₘₐₓ for 2-methyl-5-(1-methylpyrrolidin-2-yl)pyridine which is greater than the Cₘₐₓ of nicotine for a comparable pouched product containing an equal weight of nicotine. In some embodiments, a smaller amount of 2-methyl-5-(1-methylpyrrolidin-2-yl)pyridine may be utilized to achieve a comparable physiological effect to that of nicotine (i.e., the pouched products comprising 2-methyl-5-(1-methylpyrrolidin-2-yl)pyridine exhibit greater potency relative to nicotine). Without wishing to be bound by theory, it is believed that higher Cₘₐ may be due to increased Log P, decreased metabolism, or both, relative to nicotine.

In some embodiments, a pouched product of the disclosure comprising 2-methyl-5-(1-methylpyrrolidin-2-yl)pyridine when placed in the oral cavity of a user, exhibits an AUC for 2-methyl-5-(1-methylpyrrolidin-2-yl)pyridine which is similar to or greater than the AUC of nicotine for a comparable pouched product containing an equal weight of nicotine. In some embodiments, a smaller amount of 2-methyl-5-(1-methylpyrrolidin-2-yl)pyridine may be utilized to achieve a comparable physiological effect to that of nicotine (i.e., the pouched products comprising 2-methyl-5-(1-methylpyrrolidin-2-yl)pyridine exhibit greater potency relative to nicotine). Without wishing to be bound by theory, it is believed that the higher AUC may be due to increased Log P, decreased metabolism, or both, relative to nicotine.

### Preparation of the Composition

Compositions of the disclosure, regardless of format (e.g., granular, pouched, pastille, melt, chew, gum, and the like) are generally prepared by mixing a substituted 3-(1-methylpyrrolidin-2-yl)pyridine as described herein with additional composition components (e.g., water, fillers, sweeteners, binders, flavoring agents, buffers, salts, and the like). The manner by which the various components of the composition are combined may vary. As such, the overall mixture of the various components may be relatively uniform in nature. The components noted above, which may be in liquid or dry solid form, can be admixed in a pretreatment step prior to mixture with any remaining components of the composition, or simply mixed together with all other liquid or dry components.

In some embodiments, the method of preparing the composition comprises:
combining one or more fillers, one or more salts, and one or more sweeteners to form a dry blend;
combining a dilute aqueous solution of substituted 3-(1-methylpyrrolidin-2-yl)pyridine with water and optionally a humectant to form a solution;
combining the solution with the dry blend to form a mixture;
mixing the resulting mixture to form a wet blend; and
optionally, adding flavoring agent and water to the wet blend form the composition.

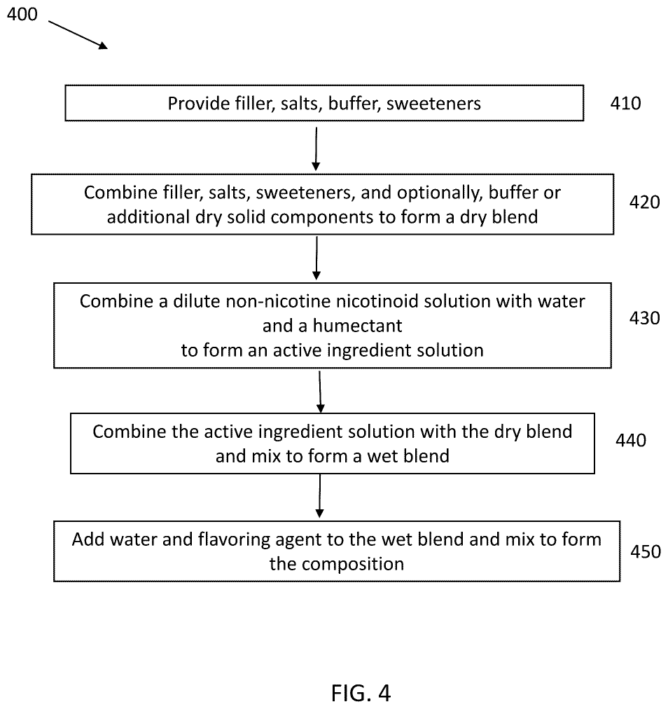

A non-limiting flow chart depicting such a method 400 of forming the composition is provided in FIG. 4. Such a method 400 is generally suitable for forming the composition in a particulate form, suitable for e.g., enclosing in a pouch to form a pouched product. With reference to FIG. 4, in some embodiments, one or more fillers, one or more salts, one or more sweeteners, and optionally a buffer, are provided 410 and combined 420 to form a dry blend.

With continued reference to FIG. 4, in some embodiments, the substituted 3-(1-methylpyrrolidin-2-yl)pyridine is provided as a dilute aqueous solution, for example, a solution in water from about 4 to about 20% by weight of the substituted 3-(1-methylpyrrolidin-2-yl)pyridine. In some embodiments, the substituted 3-(1-methylpyrrolidin-2-yl)pyridine in solution is present in the free base form. In some embodiments, the solution includes from about 8 to about 14% of free base substituted 3-(1-methylpyrrolidin-2-yl)pyridine by weight, such as from about 8, about 9, about 10, or about 11, to about 12, about 13, or about 14% by weight, based on the total weight of the solution. With continued reference to FIG. 4, in some embodiments, a dilute aqueous solution of substituted 3-(1-methylpyrrolidin-2-yl)pyridine is combined 430 with water and optionally a humectant (e.g., glycerin) to form a solution. The solution is then combined 440 with the dry blend, either by addition of the solution to the dry blend, or addition of the dry blend to the solution, and mixed to form a wet blend. Water and optional flavoring agent are then added 450 to the wet blend and mixed to form the composition.

In some embodiments, the substituted 3-(1-methylpyrrolidin-2-yl)pyridine is provided as a solid, such as solid free base or a salt or co-crystal as disclosed herein. Accordingly, in some embodiments, the substituted 3-(1-methylpyrrolidin-2-yl)pyridine in solid form is combined with the dry blend, or may be combined with water and optionally a humectant (e.g., glycerin) to form a solution. The solution is then combined 440 with the dry blend, either by addition of the solution to the dry blend, or addition of the dry blend to the solution, and mixed to form a wet blend. Water and optional flavoring agent are then added 450 to the wet blend and mixed to form the composition.

In some embodiments, the substituted 3-(1-methylpyrrolidin-2-yl)pyridine is sorbed onto a porous particulate carrier material, such as microcrystalline cellulose (MCC) prior to incorporation within a composition of the disclosure. In some embodiments, the MCC material has an average particle size range of about 15 to about 250 microns. The particulate carrier material with the sorbed substituted 3-(1-methylpyrrolidin-2-yl)pyridine can then be combined with further composition components as described herein.

In some embodiments, the substituted 3-(1-methylpyrrolidin-2-yl)pyridine is complexed with a cyclodextrin, such as beta-cyclodextrin or derivative thereof, prior to incorporation within a composition of the disclosure. For example, in some embodiments, a slurry of beta-cyclodextrin, substituted 3-(1-methylpyrrolidin-2-yl)pyridine, and water is heated to form a solution. Upon cooling, the precipitate is collected and dried to form a free-flowing powder. The ratio of beta-cyclodextrin to substituted 3-(1-methylpyrrolidin-2-yl)pyridine may vary, but is typically an approximately 1:1 molar ratio. Other cyclodextrins, such as hydroxy substituted and sulfo-beta ether cyclodextrins are also contemplated herein.

In some embodiments, the substituted 3-(1-methylpyrrolidin-2-yl)pyridine is complexed with pectin prior to incorporation within a composition of the disclosure. For example, in some embodiments, the substituted 3-(1-methylpyrrolidin-2-yl)pyridine in water is mixed with apple-derived pectin in an amount sufficient to form a thick paste.

The various components of the composition (e.g., filler, substituted 3-(1-methylpyrrolidin-2-yl)pyridine, optionally in a dilute aqueous solution, flavoring, sweeteners, salts, and the like) may be contacted, combined, or mixed together using any mixing technique or equipment known in the art. Any mixing method that brings the composition ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as models FKM130, FKM600, FKM1200, FKM2000 and FKM3000 from Littleford Day, Inc., Ploughshare types of mixer cylinders, Hobart brand mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. Manners and methods for formulating compositions will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and U.S. Pat. No. 6,077,524 to Bolder et al., each of which is incorporated herein by reference.

In some embodiments, the composition is in the form of a gum, chew, pastille, lozenge, tablet, film, or melt. Methods of preparing such forms are generally known in the art and non-limiting methods are provided in the examples below. In each instance, the method generally comprises combining a dilute aqueous solution of substituted 3-(1-methylpyrrolidin-2-yl)pyridine with other appropriate components appropriate to the particular composition.

In some embodiments, the composition is in the form of a translucent or transparent lozenge. The manners and methods used to formulate and manufacture the translucent lozenge product can vary. For example, the composition can be prepared via any method commonly used for the preparation of hard-boiled confections. Exemplary methods for the preparation of hard confections can be found, for example, in LFRA Ingredients Handbook, Sweeteners, Janet M. Dalzell, Ed., Leatherhead Food RA (December 1996), pp. 21-44, which is incorporated herein by reference. Typically, a translucent lozenge comprises substituted 3-(1-methylpyrrolidin-2-yl)pyridine, a sugar substitute, and a sugar alcohol, and may contain various additional substances (e.g., salts, preservatives, further sweeteners, water, organic acids, buffers, flavorings, and the like) as described herein. Typically, a mixture of composition components is formed and is heated until it melts. Subsequently, the mixture is heated to or past the hard crack stage. In confectionary making, the hard crack stage is defined as the temperature at which threads of the heated mixture (obtained by pulling a sample of cooled syrup between the thumb and forefinger) are brittle or as the temperature at which trying to mold the syrup results in cracking. The temperature at which the hard crack stage is achieved can vary, depending on the specific makeup of the product mixture but generally is between about 145° C. and about 170° C. Typically, the mixture is not heated above about 171° C., which is the temperature at which caramelization begins to occur. Typically, the mixture is heated to the hard crack stage temperature or above and then allowed to cool. The heating can be conducted at atmospheric pressure or under vacuum. The mixture is then formed into the desired shape. In some embodiments, the mixture is poured directly into molds, formed (e.g., rolled or pressed) into the desired shape, or extruded. If desired, the mixture can be extruded or injection molded. In certain embodiments, the mixture is formed or extruded into a mold of desired shape in an enclosed system, which may require decreased temperature and which may limit evaporation of certain mixture components. For example, such a system may limit the evaporation of volatile components including, but not limited to, flavorants. Other methods of producing lozenges are also intended to be encompassed herein.

As disclosed herein above, in some embodiments, the composition is in the form of a snuff or dip, optionally enclosed in a fleece pouch. To prepare the composition in this form, generally, the non-tobacco botanical material, typically in shredded or particulate form, is treated with a mixture comprising the other composition components, such as the active agent (e.g., substituted 3-(1-methylpyrrolidin-2-yl)pyridine,) water, salt, humectant flavoring agents, sweeteners, pH adjusting agent, buffers, coloring agents binders, and the like. The non-tobacco botanical material is shredded to a size that gives an acceptable oral chew texture, for example a mesh size from about 4 to about 40. In some embodiments, the moist snuff may be pasteurized, through heat, pressure, and/or irradiation. Additionally, preservative agents may be present, such sodium benzoate, sorbic acid, and/or essential oils. In some embodiments, the composition is enclosed in a pouch as described herein. In pouched embodiments, the non-tobacco botanical material may be shredded to a particle size conducive to pouching (such as about 16 to 64 US mesh).

Many modifications and other embodiments of the disclosure will come to mind to one skilled in the art to which this disclosure pertains having the benefit of the teachings presented in the foregoing description. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

Aspects of the present disclosure are more fully illustrated by the following examples, which are set forth to illustrate certain aspects of the present disclosure and are not to be construed as limiting thereof.

### Example 1. Preparation of a Pouched Oral Product

Pouched oral products comprising a composition according to present disclosure, including approximately 0.25 mg to 2.5 mg of 2-methyl-5-(1-methylpyrrolidin-2-yl)pyridine as the active ingredient, were prepared. The fill composition included 2-methyl-5-(1-methylpyrrolidin-2-yl)pyridine, microcrystalline cellulose (MCC), sweeteners, water, and additional components as disclosed herein (salt, humectant, buffer, flavoring agent). The fill composition had the formulation provided in Table 2.

To prepare the fill composition, a dry blend of MCC, salt, sodium bicarbonate, and sweeteners was mixed in a blender. To this dry mix was added a mixture of water, 2-methyl-5-(1-methylpyrrolidin-2-yl)pyridine solution, and propylene glycol with mixing. To the wet mix was added flavorant and additional water to provide a fill composition. Portions of the fill composition (about 440 mg) were placed in fleece pouches (about 38 mm by 12 mm). Additional water was added to the pouches to achieve a pouch weight of about 576 mg and calculated oven volatiles content of about 35% by weight.

TABLE 2
Fill composition ingredients

| Ingredient | % w/w |
|---|---|
| microcrystalline cellulose | 55-75 |
| water | 8-12 |
| 2-methyl-5-(1-methylpyrrolidin-2-yl)pyridine, 12% aq. | 0.5-5 |
| solution | |
| sweetener | 1-3 |
| sodium chloride | 1-4 |
| propylene glycol | 0.1-1.5 |
| sodium bicarbonate | 0.1-0.6 |
| flavoring agent | 0-3 |

### Example 2. Preparation of Flavored Chewing Gum

Chewing gum tablets containing a flavoring agent and approximately 0.25 mg to 2 mg 2-methyl-5-(1-methylpyrrolidin-2-yl)pyridine are prepared using the formulation components provided in Table 3.

Sorbitol, isomalt, and sodium benzoate are combined to give a dry mixture. Separately, maltitol syrup (aqueous, 75% by weight), glycerin, and 2-methyl-5-(1-methylpyrrolidin-2-yl)pyridine benzoate (aqueous, 11% by weight) solution are combined to form a liquid mixture. The gum base (Artica T, CAFOSA Gum S/A, Barcelona, Spain) is warmed in a microwave oven under low power for approximately five minutes to soften. The heated, softened gum base is mixed by hand until uniform and transferred to the bowl of a commercial mixer. Approximately ⅓ of the liquid mixture is added to the gum base followed by two minutes of mixing. Approximately ⅓ of the dry mixture, along with sucralose, is added to the gum base-liquid mixture followed by two minutes of mixing. Additions of the dry and liquid mixtures are each repeated twice, followed in each case by a further two minutes of mixing to incorporate the remaining dry and liquid mixtures. The flavoring agent is added to the mixture followed by mixing for 45 seconds. The mixture is removed from the mixer bowl and dusted with mannitol powder, then run through a sheeter to provide a uniform thickness of the composition. Square tablets weighing 1.7 g each are die punched from the composition sheet.

TABLE 3
Oral gum composition components and amounts

| Component | % w/w |
|---|---|
| gum base | 24-29 |
| sorbitol | 32-40 |
| maltitol (75% aqueous) | 13-16 |
| isomalt | 13-16 |
| glycerin | 1.4-1.7 |
| sweetener | 0.1-1 |
| flavoring agent | 0-4 |
| mannitol | 0.9-1.1 |
| 2-methyl-5-(1-methylpyrrolidin-2-yl)pyridine (11% | 0.1-1 |
| aqueous solution) | |
| sodium benzoate | 0-0.4 |

### Example 3. Preparation of Tablet

A composition in tablet form is prepared from a composition containing a mixture of fillers, about 0.25 mg to 2 mg 2-methyl-5-(1-methylpyrrolidin-2-yl)pyridine, and additional components as disclosed herein (salt, sweeteners, processing aid). Aqueous 2-methyl-5-(1-methylpyrrolidin-2-yl)pyridine benzoate (aqueous, 11% by weight) and microcrystalline cellulose were mixed. The fillers, sweetener, salt, 2-methyl-5-(1-methylpyrrolidin-2-yl)pyridine benzoate microcrystalline cellulose mixture, and processing aid are combined and mixed thoroughly. The mixture is compressed and tableted using a Fette 1200i tablet press. The tablets are optionally coated with wax and shellac. The ingredients of the composition and their concentrations in the composition in weight % are provided in Table 4. The tablets each weigh 800 mg.

TABLE 4
Tablet ingredients

| Ingredient | % w/w |
|---|---|
| Emdex ® | 50-61 |
| Isomalt | 28-34 |
| Hydroxypropyl cellulose | 7-8 |
| Sweetener | 0.1-1 |
| Sodium chloride | 0.2-0.4 |
| Sodium stearyl fumarate | 1.1-1.4 |
| Flavor | 0-1 |
| Color | 0-0.1 |
| 2-Methyl-5-(1-methylpyrrolidin-2-yl)pyridine benzoate, | 0.3-2.3 |
| 11% aqueous solution | |
| Microcrystalline cellulose | 0.5-8 |
| Sodium benzoate | 0-0.8 |
| Carnauba wax | 0-0.15 |
| Shellac | 0-0.4 |

### Example 4. Preparation of a Chewable

A composition in chewable form is prepared from a composition containing a mixture of fillers, about 0.25 mg to 2 mg 2-methyl-5-(1-methylpyrrolidin-2-yl)pyridine, and additional components as disclosed herein (salt, sweeteners, flavoring agent, water, binder, citric acid, gelation agent). The ingredients of the composition and their concentrations in the composition in weight % are provided in Table 5.

The pectin binder is pre-blended with a portion of the isomalt. Water is added, and the mixture heated to boiling with stirring. Maltitol syrup (aqueous, 75% by weight) and any remaining isomalt are added to the boiling mixture, followed by trisodium citrate. The mixture is cooked to 78 brix. Heat is removed, and sweetener (e.g., sucralose and acesulfame K, colorant and flavorant are added, along with the citric acid and dicalcium phosphate and 2-methyl-5-(1-methylpyrrolidin-2-yl)pyridine, the mixture thoroughly combined, and the composition deposited into starch molds for storage at ambient temperature. The chews each weigh 2600 mg.

TABLE 5
Chewable ingredients

| Ingredient | % w/w |
|---|---|
| Isomalt | 12-20 |
| Maltitol syrup, 75% aqueous | 48-72 |
| solution | |
| 2-methyl-5-(1-methylpyrrolidin-2- | 0.1-0.7 |
| yl)pyridine, 12% aqueous solution | |
| Water | 12-18 |
| Sodium benzoate | 0-0.3 |
| Dicalcium phosphate | 0.4-0.6 |
| Citric acid | 0.5-1.5 |
| Trisodium citrate | 0.5-1.5 |
| Flavor | 0-0.9 |
| Pectin | 1-2 |
| Sweetener | 0.05-0.5 |
| Color | 0.05-0.15 |

### Example 5. Preparation of a Meltable

A composition in meltable form is prepared from a composition containing a filler, a lipid, about 0.25 mg to 2 mg 2-methyl-5-(1-methylpyrrolidin-2-yl)pyridine, and additional components as disclosed herein (salt, sweeteners, flavoring agent). The ingredients of the composition and their concentrations in the composition in weight % are provided in Table 6.

A portion of the palm oil is melted and mixed with the isomalt in a mixer. The mixture is transferred to a dry roll mill and milled until the particle size is less than 20 microns. In a mixer, the milled isomalt-palm oil is combined with the remaining portion of palm oil. The base is warmed to a fluid consistency. Sunflower oil, the dry ingredients, 2-methyl-5-(1-methylpyrrolidin-2-yl)pyridine and flavor are mixed in. The isomalt-palm oil-ingredient mixture is transferred to a heated depositing funnel. The appropriate weight of the samples is deposited into a shape mold. If needed, the mold is placed on a vibrator to ensure even filling. The product is allowed to cool and solidify, then removed from the mold. The melts each weigh 1300 mg.

TABLE 6
Meltable ingredients

| Ingredient | % w/w |
|---|---|
| Isomalt | 35-55 |
| Lipid (e.g., palm oil) | 32-48 |
| 2-methyl-5-(1- | 0.15-1.3 |
| methylpyrrolidin-2- | |
| yl)pyridine, 12% | |
| aqueous solution | |
| Sodium benzoate | 0-0.6 |
| Sodium chloride | 0.5-1.5 |
| Sunflower lecithin | 0.25-0.5 |
| Sunflower oil | 2-3.5 |
| Sweetener | 0.05-0.5 |
| Flavor | 0-1.5 |

### Example 6. Preparation of a Pastille

An oral product in the form of a pastille, containing about 0.25 mg to 2 mg 2-methyl-5-(1-methylpyrrolidin-2-yl)pyridine, is prepared. An aqueous mixture is prepared. The aqueous mixture is formed by admixing water, a salt, a sweetener (e.g., sucralose), a humectant (e.g., glycerin), sodium benzoate, and a flavoring agent. Next, an aqueous gum Arabic solution (50% by weight) is heated to a temperature of about 71° C. and a solution of 2-methyl-5-(1-methylpyrrolidin-2-yl)pyridine is stirred into the heated gum component. The heated gum is then added to the aqueous composition to form a mixture. Then, sugar alcohols (e.g., isomalt and maltitol) are heated to a temperature of about 175° C. and then cooled to a temperature of about 150° C. The cooled sugar alcohol is then added to the mixture and stirred in to form a pastille composition which is allowed to cool.

The pastille composition is then heated to about 71° C. and deposited into a starch mold. The pastille composition remains in the starch mold for about 24 hours at about 60° C. The pastille composition is allowed to cool and then removed from the starch mold. The oral composition is then cured at ambient room temperature for about 24 hours to provide the pastille product weighing about 2000 mg each. The pastilles are optionally coated with an anti-stick agent (e.g., MCT oil or Capol®). Table 7 below illustrates the relative percentages of each individual component in the pastille.

TABLE 7
Pastille ingredients

| Ingredient | % (w/w) |
|---|---|
| Gum Arabic, 50% aqueous solution | 48-59 |
| Glycerin | 1.8-2.2 |
| Sodium Chloride | 1.8-2.2 |
| Sweetener | 0.1-1 |
| Flavor | 0-1.5 |
| Isomalt | 29-35 |
| Maltitol, 75% aqueous solution | 5.3-6.4 |
| 2-Methyl-5-(1-methylpyrrolidin-2-yl)pyridine | 0.1-0.9 |
| benzoate, 11% aqueous solution | |
| Sodium Benzoate | 0-0.3 |
| Water | 1.5-1.8 |
| Color | 0-0.1 |
| Anti-stick agent | 0-0.1 |

### Example 7. Preparation of a Translucent Lozenge

An oral product in the form of a translucent lozenge is prepared including about 0.25 mg to 2 mg 2-methyl-5-(1-methylpyrrolidin-2-yl)pyridine. Isomalt and maltitol syrup are heated to melting without stirring, to hard crack temperature (e.g., 165° C.). The melt is cooled to approximately 143° C. with very slight, discontinuous stirring. Other components (2-methyl-5-(1-methylpyrrolidin-2-yl)pyridine benzoate (aqueous, 11% solution), sodium benzoate, salts, sweeteners, and/or flavorants) are added to the mixture and folded in carefully with a spatula without introducing much air into the melt. The melt is further cooled to about 120° C. The mixture is poured into a glass beaker, the contents are poured from the beaker in a chord on a metal brick, in breakaway molds with niches of suitable dimensions, or in molds (for individual lozenges, weighing about 2000 mg each). Optionally, the mixture is reheated in order to obtain a suitable viscosity of the melt. The melt is cooled for a time suitable for solidifying (e.g., at room temperature). The lozenges are optionally coated with an anti-stick agent (e.g., MCT oil, or Capol®). Table 8 below illustrates the relative percentages of each individual component in the lozenge.

TABLE 8
Lozenge ingredients

| Ingredient | % (w/w) |
|---|---|
| Isomalt | 86-96 |
| Maltitol, 75% aqueous solution | 0.9-1.1 |
| Sodium Chloride | 1.2-1.7 |
| Sweetener | 0-0.1 |
| Flavor | 0-2 |
| Anti-stick agent | 0-0.2 |
| 2-Methyl-5-(1-methylpyrrolidin-2-yl)pyridine | 0.1-0.9 |
| benzoate, 11% aqueous solution | |
| Sodium benzoate | 0-0.3 |

### Example 8. Preparation of a Pouched Oral Product

A pouched oral product comprising a fill composition according to present disclosure comprising 5 mg 2-methyl-5-(1-methylpyrrolidin-2-yl)pyridine as the active ingredient was prepared. The fill composition included 2-methyl-5-(1-methylpyrrolidin-2-yl)pyridine, microcrystalline cellulose (MCC), sweeteners, water, and additional components as disclosed herein (salt, humectant, flavoring agent, ion pairing agent). The fill composition had the formulation provided in Table 9.

To prepare the fill composition, a dry blend of MCC, salt, about half the total sodium benzoate amount, and the sweeteners were mixed in a blender. To this dry mix was added a mixture of water, 2-methyl-5-(1-methylpyrrolidin-2-yl)pyridine solution, the remaining sodium benzoate, flavor agent, and propylene glycol with mixing.

Portions of the fill composition (about 440 mg) were placed in fleece pouches (about 38 mm by 12 mm). Additional water was added to the pouches to achieve a pouch weight of about 720 mg and calculated oven volatiles content of about 48% by weight.

Portions of the fill composition (about 440 mg) were placed in fleece pouches (about 38 mm by 12 mm). Additional water was added to the pouches to achieve a pouch weight of about 576 mg and calculated oven volatiles content of about 35% by weight.

TABLE 9
Fill composition ingredients

| Ingredient | % w/w |
|---|---|
| microcrystalline cellulose | 68% |
| water | 2.6% |
| 2-methyl-5-(1- | 0.5% |
| methylpyrrolidin-2- | |
| yl)pyridine, 12% aq. | |
| solution | |
| 2-methyl-5-(1- | 9.7% |
| methylpyrrolidin-2- | |
| yl)pyridine benzoate, 11% | |
| aq. solution | |
| sweetener | 3.4% |
| salt (e.g., sodium chloride) | 2.6% |
| propylene glycol | 7.8% |
| sodium benzoate | 3.6% |
| flavor | 1.0% |

### Example 9. Preparation of a Pouched Oral Product-Premix Method

Pouched oral products comprising compositions according to present disclosure including 1 mg to 3 mg 2-methyl-5-(1-methylpyrrolidin-2-yl)pyridine as the active ingredient were prepared. The compositions included 2-methyl-5-(1-methylpyrrolidin-2-yl)pyridine, microcrystalline cellulose (MCC), sweeteners, water, and additional components as disclosed herein (salt, humectant, flavoring agent, ion pairing agent). The premix fill composition had the formulation provided in Table 10A.

To prepare the premix fill composition, a dry blend of MCC, salt, sodium benzoate, and sweeteners was mixed in a blender. To this dry mix was added a mixture of water, 2-methyl-5-(1-methylpyrrolidin-2-yl)pyridine solution, sodium benzoate, and propylene glycol with mixing. To the wet mix was added flavorant, propylene glycol, additional water, optional sodium bicarbonate solution, to provide a final fill composition provided in Table 10B. Portions of the fill composition (about 440 mg) were placed in fleece pouches (about 38 mm by 12 mm). Additional water was added to the pouches to achieve a pouch weight of about 720 mg and calculated oven volatiles content of about 48% by weight.

Portions of the fill composition (about 440 mg) were placed in fleece pouches (about 38 mm by 12 mm). Additional water was added to the pouches to achieve a pouch weight of about 576 mg and calculated oven volatiles content of about 35% by weight.

TABLE 10A
Premix fill composition ingredients

| Ingredient | % w/w |
|---|---|
| microcrystalline cellulose | 73-72 |
| water | 6-10 |
| 2-methyl-5-(1- | 0.8-1.3 |
| methylpyrrolidin-2- | |
| yl)pyridine, 12% aq. | |
| solution | |
| sweetener | 3-4 |
| sodium chloride | 3.3-4 |
| propylene glycol | 6.5-7 |
| sodium benzoate | 0-1.2 |

TABLE 10B
Fill composition ingredients

| Ingredient | % w/w |
|---|---|
| Premix | 97 |
| Water | 0.5 |
| Propylene Glycol | 1-2.3 |
| Flavor agent | 0-0.6 |

### Example 10. Preparation of a Dry Pouched Oral Product

A pouched oral product having a low moisture level and comprising a composition according to the present disclosure was prepared. The composition included 2-methyl-5-(1-methylpyrrolidin-2-yl)pyridine as the active ingredient, with each pouch comprising 1 mg of the 2-methyl-5-(1-methylpyrrolidin-2-yl)pyridine. The composition further included microcrystalline cellulose (MCC), sweeteners, water, and additional components as disclosed herein (salt, humectant, buffer, flavoring agent). The composition had the formulation provided in Table 11. To prepare the composition, a blend of 2-methyl-5-(1-methylpyrrolidin-2-yl)pyridine (aqueous solution of free base and aqueous solution of benzoate, 12% and 11% by weight, respectively), MCC, salt, maltitol, hydroxypropyl cellulose, sodium benzoate, sweeteners and flavoring agent was mixed in a blender.

Portions of the fill composition (about 370 mg) were placed in fleece pouches (about 30 mm by 12 mm). Additional water was added to the pouches to achieve a pouch weight of about 400 mg and calculated oven volatiles content of about 4% by weight.

TABLE 11
Fill composition ingredients

| Ingredient | % w/w |
|---|---|
| microcrystalline cellulose | 30-42 |
| 2-methyl-5-(1-methylpyrrolidin-2-yl)pyridine, free | 1.4 |
| base, 12% aqueous solution | |
| 2-methyl-5-(1-methylpyrrolidin-2-yl)pyridine | 0.9 |
| benzoate, 11% aqueous solution | |
| maltitol | 54 |
| sweetener | 0.03-0.3 |
| sodium chloride | 1-3 |
| sodium benzoate | 0.5-1.5 |
| hydroxypropyl cellulose | 2-4 |
| flavoring agent | 0-3 |

### Example 11. Preparation of a Pouched Oral Product-Spherical MCC

A pouched oral product comprising a fill composition according to the present disclosure was prepared. The composition included 2-methyl-5-(1-methylpyrrolidin-2-yl)pyridine as the active ingredient, with each pouch comprising about 0.25 mg of the 2-methyl-5-(1-methylpyrrolidin-2-yl)pyridine. The composition further included spherical microcrystalline cellulose (MCC), sweeteners, water, and additional components as disclosed herein (salt, humectant, buffer, flavoring agent). The composition had the formulation provided in Table 12. To prepare the fill composition, a blend of 2-methyl-5-(1-methylpyrrolidin-2-yl)pyridine (aqueous solution of monomenthyl succinate salt, 5% by weight), water, spherical MCC (JRS 350), salt, sodium bicarbonate, propylene glycol, sweeteners, and flavoring agent was mixed in a blender. Portions of the fill composition (about 416 mg) were placed in fleece pouches (about 30 mm by 12 mm). Additional water was added to the pouches to achieve a pouch weight of about 509 mg and calculated oven volatiles content of about 23% by weight. The pouch pH was about 7.5.

TABLE 12
Fill composition ingredients

| Ingredient | % w/w |
|---|---|
| Spherical microcrystalline cellulose | 72-90 |
| water | 5-10 |
| 2-methyl-5-(1-methylpyrrolidin-2-yl)pyridine | 1.2 |
| monomenthyl succinate, 5% aq. solution | |
| sweeteners | 0-4.5 |
| sodium chloride | 2-4.5 |
| propylene glycol | 0.2-0.4 |
| sodium bicarbonate | 0.2-1 |
| flavoring agent | 0-3 |

### Example 12. Preparation of a Pouched Oral Product-Spherical MCC

A pouched oral product comprising a fill composition according to the present disclosure was prepared. The composition included 2-methyl-5-(I-methylpyrrolidin-2-yl)pyridine as the active ingredient, with each pouch comprising 0.25 mg of the 2-methyl-5-(1-methylpyrrolidin-2-yl)pyridine. The composition further included spherical microcrystalline cellulose (MCC), sweeteners, water, and additional components as disclosed herein (salt, propylene glycol, sodium benzoate, flavoring agent). The composition had the formulation provided in Table 13. To prepare the fill composition, a blend of 2-methyl-5-(1-methylpyrrolidin-2-yl)pyridine (aqueous solution of free base, 12% by weight), water, spherical MCC, salt, sodium benzoate, sodium bicarbonate, propylene glycol, sweeteners and flavoring agent was mixed in a blender. Portions of the fill composition (about 416 mg) were placed in fleece pouches (about 30 mm by 12 mm). Additional water was added to the pouches to achieve a pouch weight of about 509 mg and calculated oven volatiles content of about 23% by weight.

TABLE 13
Fill composition ingredients

| Ingredient | % w/w |
|---|---|
| Spherical microcrystalline cellulose | 72-90 |
| water | 5-10 |
| 2-methyl-5-(1-methylpyrrolidin-2-yl)pyridine, | 0.5 |
| 12% aq. solution | |
| sweetener | 0-4.5 |
| sodium chloride | 2-4 |
| propylene glycol | 0.2-0.4 |
| sodium benzoate | 0.2-0.3 |
| sodium bicarbonate | 0-1 |
| flavoring agent | 0-3 |

### Example 13. Preparation of a Pouched Oral Product-Pectinate Complex

A pouched oral product comprising a fill composition according to the present disclosure was prepared. The composition included 2-methyl-5-(1-methylpyrrolidin-2-yl)pyridine as the active ingredient, with each pouch comprising 1 mg of the 2-methyl-5-(1-methylpyrrolidin-2-yl)pyridine. The composition further included microcrystalline cellulose (MCC), sweeteners, water, and additional components as disclosed herein (salt, propylene glycol, sodium benzoate, flavoring agent). The fill composition had the formulation provided in Table 14. A pectinate complex was first prepared by combining 309 g of 2-methyl-5-(1-methylpyrrolidin-2-yl)pyridine with 60 g of low methoxy amidated apple pectin to form a thick paste then combined with MCC to form a dry powder containing 2% by weight of 2-methyl-5-(1-methylpyrrolidin-2-yl)pyridine.

To prepare the composition, a blend of 2-methyl-5-(1-methylpyrrolidin-2-yl)pyridine pectinate complex and MCC mixture, water, MCC, salt, sodium benzoate, propylene glycol, sodium bicarbonate, sweeteners and flavoring agent was mixed in a blender. Portions of the fill composition (about 440 mg) were placed in fleece pouches (about 38 mm by 12 mm). Additional water was added to the pouches to achieve a pouch weight of about 720 mg and calculated oven volatiles content of about 49% by weight. The pouch pH was about 7.2.

TABLE 14
Fill composition ingredients

| Ingredient | % w/w |
|---|---|
| Microcrystalline cellulose (MCC) | 50-70 |
| Water | 10-20 |
| 2-Methyl-5-(1-methylpyrrolidin-2-yl)pyridine pectinate | 10.7 |
| complex and MCC mixture (2% of | |
| 2-methyl-5-(1-methylpyrrolidin-2-yl)pyridine) | |
| Sweetener | 0-4.5 |
| Sodium chloride | 2-4 |
| Propylene glycol | 0.5-1.5 |
| Sodium bicarbonate | 0.02-0.2 |
| Sodium benzoate | 0.9 |
| Flavor | 0-3 |

### Example 14. Preparation of a Pouched Oral Product-HCl Mixture

A pouched oral product comprising a fill composition according to the present disclosure was prepared. The composition included 2-methyl-5-(1-methylpyrrolidin-2-yl)pyridine as the active ingredient, with each pouch comprising 1 mg of the 2-methyl-5-(1-methylpyrrolidin-2-yl)pyridine. The composition further included microcrystalline cellulose (MCC), sweeteners, water, and additional components as disclosed herein (salt, propylene glycol, sodium benzoate, flavoring agent). The fill composition had the formulation provided in Table 15. A 2-methyl-5-(1-methylpyrrolidin-2-yl)pyridine hydrochloric acid solution was first prepared from 48 g 12% aqueous 2-methyl-5-(1-methylpyrrolidin-2-yl)pyridine, 0.32 g concentrated hydrochloric acid (about 37% by weight), and 9 g water. To prepare the composition, a blend of the aqueous solution of 2-methyl-5-(1-methylpyrrolidin-2-yl)pyridine and hydrochloric acid (10% by weight), water, MCC, salt, sodium benzoate, propylene glycol, sweeteners and flavoring agent was mixed in a blender. About 440 mg portions of the fill composition were placed in fleece pouches (about 38 mm by 12 mm). Additional water was added to the pouches to achieve a pouch weight of about 720 mg and calculated oven volatiles content of about 48% by weight. The pouch pH was about 6.3.

TABLE 15
Fill composition ingredients

| Ingredient | % w/w |
|---|---|
| Microcrystalline cellulose | 65-75 |
| water | 10-20 |
| 2-Methyl-5-(1-methylpyrrolidin-2-yl)pyridine | 2.3 |
| hydrochloric acid mixture, 10% aq. solution | |
| Sweetener | 0-4.5 |
| Sodium chloride | 2-4 |
| Propylene glycol | 0.5-1.5 |
| Sodium benzoate | 0.9 |
| Flavor | 0-3 |

### Example 15. Preparation of a Pouched Oral Product

A pouched oral product comprising a fill composition according to the present disclosure was prepared. The composition included 2-methyl-5-(1-methylpyrrolidin-2-yl)pyridine as the active ingredient, with each pouch comprising 0.5 mg of the 2-methyl-5-(1-methylpyrrolidin-2-yl)pyridine. The composition further included microcrystalline cellulose (MCC), sweeteners, water, and additional components as disclosed herein (salt, humectant, sodium benzoate, sodium bicarbonate solution, flavoring agent). The fill composition had the formulation provided in Table 16. To prepare the composition, a blend of 2-methyl-5-(1-methylpyrrolidin-2-yl)pyridine (aqueous solution of free base, 12% by weight), water, MCC, salt, sodium benzoate, sodium bicarbonate (4% aqueous solution), propylene glycol, sweeteners and flavoring agent was mixed in a blender. Portions of the fill composition (about 440 mg) were placed in fleece pouches (about 38 mm by 12 mm). Additional water was added to the pouches to achieve a pouch weight of about 720 mg and calculated oven volatiles content of about 48% by weight.

TABLE 16
Fill composition ingredients

| Ingredient | % w/w |
|---|---|
| microcrystalline cellulose | 55-75 |
| water | 8-12 |
| 2-methyl-5-(1-methylpyrrolidin-2-yl)pyridine, | 1.2 |
| 12% aq. solution | |
| sweetener | 1.5-3.5 |
| salt (e.g., sodium chloride) | 2-4 |
| Sodium bicarbonate (4% aq) | 0.4-0.6 |
| propylene glycol | 0.5-1.5 |
| sodium benzoate | 0.8-1.0 |
| flavoring agent | 2-3 |

### Example 16. Preparation of a Pouched Oral Product

A pouched oral product comprising a fill composition according to the present disclosure was prepared. The composition included 2-methyl-5-(1-methylpyrrolidin-2-yl)pyridine as the active ingredient, with each pouch comprising 1 mg of the 2-methyl-5-(1-methylpyrrolidin-2-yl)pyridine. The fill composition further included microcrystalline cellulose (MCC), sweeteners, water, and additional components as disclosed herein (salt, humectant, sodium benzoate, flavoring agent). The composition had the formulation provided in Table 17.

To prepare the composition, a dry blend of MCC, sodium chloride, and sweeteners was prepared by combining the components in a mixer. Separately, a wet blend was prepared by combining water, 2-methyl-5-(1-methylpyrrolidin-2-yl)pyridine (aqueous solution of free base, 12% by weight), 2-methyl-5-(1-methylpyrrolidin-2-yl)pyridine (aqueous solution of benzoate salt, 11% by weight), propylene glycol, and sodium benzoate was mixed in a blender. The wet blend, dry blend, and flavor were combined and mixed in a blender to give the fill composition. Portions of the fill composition (about 440 mg) were placed in fleece pouches (about 38 mm by 12 mm). Additional water was added to the pouches to achieve a pouch weight of about 720 mg and calculated oven volatiles content of about 48% by weight. The pouch pH was about 6.

TABLE 17
Fill composition ingredients

| Ingredient | % w/w |
|---|---|
| Microcrystalline cellulose | 55-75 |
| Water | 8-12 |
| 2-Methyl-5-(1-methylpyrrolidin-2- | 1.2 |
| yl)pyridine, 12% aq. solution | |
| 2-Methyl-5-(1-methylpyrrolidin-2-yl)pyridine | 0.7 |
| benzoate, 11% aq. solution | |
| Sweetener | 0.1-5 |
| Sodium chloride | 2.5-4.5 |
| Propylene glycol | 6-9 |
| Sodium benzoate | 0.8 |
| Flavor | 0-3 |

### Example 17. Preparation of a Pouched Oral Product

A pouched oral product comprising a fill composition according to the present disclosure was prepared. The composition included 2-methyl-5-(1-methylpyrrolidin-2-yl)pyridine as the active ingredient, with each pouch comprising 1 mg of the 2-methyl-5-(1-methylpyrrolidin-2-yl)pyridine. The composition further included microcrystalline cellulose (MCC), sweeteners, water, and additional components as disclosed herein (salt, humectant, flavoring agent). The fill composition had the formulation provided in Table 18. To prepare the composition, a dry blend of MCC, sodium chloride, and sweeteners was prepared by combining the components in a mixer. Separately, a wet blend was prepared by combining water, 2-methyl-5-(1-methylpyrrolidin-2-yl)pyridine (aqueous solution of free base, 12% by weight), 2-methyl-5-(1-methylpyrrolidin-2-yl)pyridine (aqueous solution of benzoate salt, 11% by weight), propylene glycol, and sodium benzoate was mixed in a blender. The wet blend, dry blend, and flavor were combined and mixed in a blender to give the fill composition. Portions of the fill composition (about 323 mg) were placed in fleece pouches (about 30 mm by 12 mm). Additional water was added to the pouches to achieve a pouch weight of about 421 mg and calculated oven volatiles content of about 35% by weight. The pouch pH was about 6.4.

TABLE 18
Fill composition ingredients

| Ingredient | % w/w |
|---|---|
| Microcrystalline cellulose | 55-75 |
| Water | 8-12 |
| 2-Methyl-5-(1-methylpyrrolidin-2-yl)pyridine, | 1.7 |
| 12% aq. solution | |
| 2-Methyl-5-(1-methylpyrrolidin-2-yl)pyridine | 1 |
| benzoate, 11% aq. solution | |
| Sweetener | 0.1-4 |
| Sodium chloride | 2-3.5 |
| Propylene glycol | 6-9 |
| Sodium benzoate | 0.8 |
| Flavor | 0-3 |

### Example 18. Preparation of a Pouched Oral Product

A pouched oral product comprising a fill composition according to the present disclosure was prepared. The composition included 2-methyl-5-(1-methylpyrrolidin-2-yl)pyridine as the active ingredient, with each pouch comprising 3 mg of the 2-methyl-5-(1-methylpyrrolidin-2-yl)pyridine, The composition further included microcrystalline cellulose (MCC), sweeteners, water, and additional components as disclosed herein (salt, humectant, sodium benzoate, flavoring agent). The fill composition had the formulation provided in Table 19. To prepare the composition, a dry blend of MCC, sodium chloride, sweeteners, and about half the total sodium benzoate was prepared by combining the components in a mixer. Separately, a wet blend was prepared by combining water, 2-methyl-5-(1-methylpyrrolidin-2-yl)pyridine (aqueous solution of free base, 12% by weight), 2-methyl-5-(1-methylpyrrolidin-2-yl)pyridine (aqueous solution of benzoate salt, 11% by weight), propylene glycol, and the remaining sodium benzoate was mixed in a blender. The wet blend, dry blend, and flavor were combined and mixed in a blender to give the fill composition. Portions of the fill composition (about 323 mg) were placed in fleece pouches (about 30 mm by 12 mm). Additional water was added to the pouches to achieve a pouch weight of about 426 mg and calculated oven volatiles content of about 35% by weight.

TABLE 19
Fill composition ingredients

| Ingredient | % w/w |
|---|---|
| Microcrystalline cellulose | 55-75 |
| Water | 3-5 |
| 2-Methyl-5-(1-methylpyrrolidin-2- | 1.2 |
| yl)pyridine, 12% aq. solution | |
| 2-Methyl-5-(1-methylpyrrolidin-2-yl)pyridine | 7.1 |
| benzoate, 11% aq. solution | |
| Sweetener | 0.1-1 |
| Sodium chloride | 1-2 |
| Xylitol | 1-3 |
| Propylene glycol | 6-9 |
| Sodium benzoate | 3.0 |
| Flavor | 0-3 |

### Example 19. Preparation of a Pouched Oral Product

A pouched oral product comprising a fill composition according to the present disclosure was prepared. The composition included 2-methyl-5-(1-methylpyrrolidin-2-yl)pyridine as the active ingredient, with each pouch comprising 1 mg of the 2-methyl-5-(1-methylpyrrolidin-2-yl)pyridine. The composition further included microcrystalline cellulose (MCC), sweeteners, water, sodium benzoate, and additional components as disclosed herein (salt, sweetener, humectant, flavoring agent). The fill composition had the formulation provided in Table 20. To prepare the composition, a dry blend of MCC, sodium chloride, and sweeteners was prepared by combining the components in a mixer. Separately, a wet blend was prepared by combining water, 2-methyl-5-(1-methylpyrrolidin-2-yl)pyridine (aqueous solution of free base, 12% by weight), 2-methyl-5-(1-methylpyrrolidin-2-yl)pyridine (aqueous solution of benzoate salt, 11% by weight), propylene glycol, and sodium benzoate was mixed in a blender. The wet blend, dry blend, and flavor were combined and mixed in a blender to give the fill composition. Portions of the fill composition (about 440 mg) were placed in fleece pouches (about 38 mm by 12 mm). Additional water was added to the pouches to achieve a pouch weight of about 720 mg and calculated oven volatiles content of about 48% by weight.

TABLE 20
Fill composition ingredients

| Ingredient | % w/w |
|---|---|
| Microcrystalline cellulose | 55-75 |
| Water | 8-10 |
| 2-Methyl-5-(1-methylpyrrolidin-2- | 1.2 |
| yl)pyridine, 12% aq. solution | |
| 2-Methyl-5-(1-methylpyrrolidin-2-yl)pyridine | 0.74 |
| benzoate, 11% aq. solution | |
| Sweetener | 0.1-3.5 |
| Sodium chloride | 3-6 |
| Propylene glycol | 6-9 |
| Sodium benzoate | 0.8 |
| Flavor | 0-3 |

### Example 20. Preparation of Pouched Oral Products and Dissolution Study Thereof

Pouched oral products comprising fill compositions according to the present disclosure were prepared. The fill compositions included 2-methyl-5-(1-methylpyrrolidin-2-yl)pyridine as the active ingredient, with each pouch comprising 0.5 mg, 1 mg, or 2 mg of the 2-methyl-5-(1-methylpyrrolidin-2-yl)pyridine. The compositions further included microcrystalline cellulose (MCC), sweeteners, water, and one or more additional components as disclosed herein (e.g., salt, sweetener, humectant, flavoring agent). A reference pouched product (Example 20E) comprising nicotine (5.5 mg) was also prepared. A citrate buffered composition (Example 20G) was also prepared using a pH 6.3 buffer containing trisodium citrate and citric acid (0.5M in citrate). Additional samples (Examples 20H and 20I) were also prepared in the same manner as Example 20G but containing different citrate concentrations. The pouched products were each prepared as in Example 19, using the fill formulations provided in Table 21.

TABLE 21
Pouch fill composition ingredients

| | Example # | | | | | | | | |
|---|---|---|---|---|---|---|---|---|---|
| | 20A | 20B | 20C | 20D | 20E (ref) | 20F | 20G | 20H | 20I |
| Component | % by weight | | | | | | | | |
| nicotine benzoate | 0 | 0 | 0 | 0 | 0.5 | 0 | 0 | 0 | 0 |
| 2-methyl-5-(1- | 1 | 1 | 4 | 4 | 0 | 1.2 | 0 | 0.0 | 0.0 |
| methylpyrrolidin- | | | | | | | | | |
| 2-yl)pyridine free | | | | | | | | | |
| base (12% aq. | | | | | | | | | |
| solution) | | | | | | | | | |
| 2-methyl-5-(1- | 0 | 0 | 0 | 0 | 0 | 0.74 | 2 | 2.1 | 2.1 |
| methylpyrrolidin- | | | | | | | | | |
| 2-y1)pyridine | | | | | | | | | |
| benzoate (11% | | | | | | | | | |
| aq. solution) | | | | | | | | | |
| microcrystalline | 72.5 | 72.5 | 71.6 | 71.6 | 67.5 | 71.6 | 67.8 | 70.5 | 70.5 |
| cellulose | | | | | | | | | |
| water | 18.5 | 18.5 | 15.2 | 15.2 | 3.3 | 9.7 | 0 | 2.8 | 6.7 |
| sweetener | 3.2 | 3.2 | 3.2 | 3.2 | 2.4 | 3.3 | 3.4 | 3.5 | 3.5 |
| sodium chloride | 3.5 | 3.5 | 3.5 | 3.5 | 3.3 | 3.8 | 3.7 | 3.9 | 3.9 |
| propylene glycol | 0.12 | 0.12 | 0.46 | 0.46 | 7.8 | 7.9 | 7.9 | 8.23 | 8.22 |
| benzoic acid | 0.07 | 0.07 | 0.29 | 0.29 | 0^{#} | 0^{#} | 0^{#} | 0^{#} | 0^{#} |
| sodium benzoate | 0.23 | 0.23 | 0.93 | 0.93 | 4.3 | 0.82 | 0.7 | 0.73 | 0.73 |
| trisodium citrate, | 0 | 0 | 0 | 0 | 0 | 0 | 13.6 | 7.36 | 3.48 |
| 0.5M (17% w/w) | | | | | | | | | |
| flavoring agent | 0.91 | 0.91 | 0.91 | 0.91 | 1.0 | 0.87 | 0.87 | 0.91 | 0.91 |
| * | 35 | 49 | 35 | 49 | 48 | 48 | 49 | 48 | 48 |

* calculated oven volatile content after overspray water was added to pouches
^{#}Does not reflect benzoic acid present as benzoate salt or generated from benzoate salt in situ

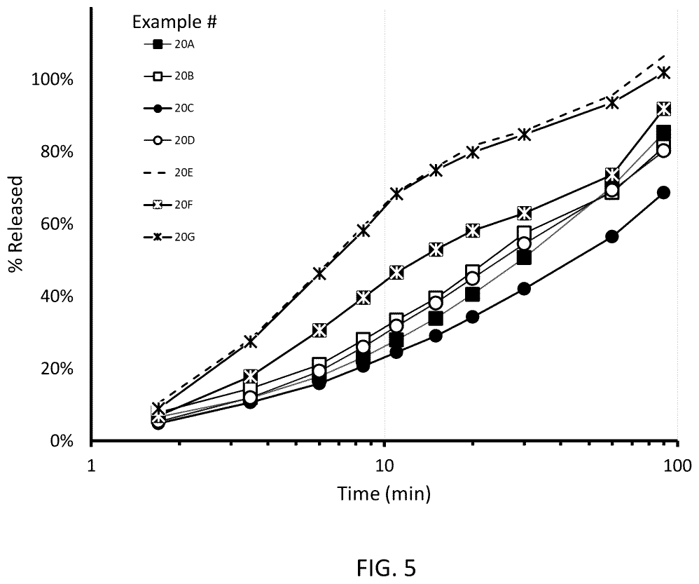

For the dissolution testing, pouched products were individually placed in 500 mL of 12 mM ammonium phosphate pH 7.4 buffer in a basket apparatus at 37C bath temperature. The baskets were stirred at 50 RPM for 60 minutes and 250 RPM for 30 minutes. Samples were taken at 1.42, 3.5, 6, 8.5, 11, 5, 20, 30, 60, and 90 minutes and analyzed for 2-methyl-5-(1-methylpyrrolidin-2-yl)pyridine concentration. The results were expressed as percent of total amount of 2-methyl-5-(1-methylpyrrolidin-2-yl)pyridine present in each pouch, and the analyses were performed in triplicate. Results are provided in FIG. 5 as % release at each time point, with the time points presented in log scale to enhance linearity.

With reference to FIG. 5, the samples released from about 70 to about 100% of the total 2-methyl-5-(1-methylpyrrolidin-2-yl)pyridine present after 90 minutes, and the release was generally linear over time. With continued reference to FIG. 5, it was surprisingly found that addition of citrate buffer (Example 20G, including 4.8 molar equivalents of benzoate and 4.5% of 0.5M citrate) increases dissolution rate and gives a dissolution profile similar to that of a reference ion-paired nicotine formulation sample (Example 20E; 4.8 molar equivalents of benzoate). With continued reference to FIG. 5, the data suggests that the dissolution rate may be increased with a higher concentration of benzoate (4.8 molar equivalents of total benzoate versus 3.2 molar equivalents of total benzoate; Example 20F versus Examples 20A through 20D). It is believed that this effect is more pronounced with formulations having a lower concentration of 2-methyl-5-(1-methylpyrrolidin-2-yl)pyridine. Without wishing to be bound by theory, it is believed that acidic sites in the fleece pouch material, and potentially in MCC, may interact with and sequester a portion of the basic 2-methyl-5-(1-methylpyrrolidin-2-yl)pyridine and increasing ionic strength in the presence of benzoate and/or citrate may help release 2-methyl-5-(1-methylpyrrolidin-2-yl)pyridine from the fleece or may reduce migration of the 2-methyl-5-(1-methylpyrrolidin-2-yl)pyridine into the fleece from the formulation. Accordingly, the concentration of ion pairing agents (e.g., benzoate) and/or buffers such as citrate, acetate, carbonate, phosphate, and the like may be varied to modulate the release properties of the pouched products with respect to 2-methyl-5-(1-methylpyrrolidin-2-yl)pyridine. Such modulation may provide a more gradual or more rapid release profile, customizing the user experience.

Samples of Examples 20F, 20G, 20H, and 20I were evaluate for Log P, composition pH, and pouch pH. The data is presented in Table 22, which show that adding 0.5M citrate buffer to compositions of 2-methyl-5-(1-methylpyrrolidin-2-yl)pyridine allowed for a closer pH value between pouch fill and finished pouched product. Surprisingly, the presence of citrate buffer had little influence on the log P of the pouch. The results are surprising because the citrate anion was expected to compete with benzoate anion in the formation of an ion pair complex; instead, it was found that a benzoate ion pair complex can still be formed in the presence of citrate.

TABLE 22
LogP and pH values

| Sample | % 0.5M Citrate | Log P | Fill pH | Pouch pH |
|---|---|---|---|---|
| 20F | 0 | 0.44 | 7.42 | 6.14 |
| 20G | 4.50 | 0.36 | 6.44 | 6.33 |
| 20H | 8.29 | 0.41 | 6.56 | 6.52 |
| 20I | 2.13 | 0.33 | 6.14 | 6.03 |

### Example 21. Preparation of Oral Strip Products

An oral product in the form of a film strip is prepared using the formulation provided in Table 23. A solution of glyceryl monostearte and Polyoxyl 40 stearate (emulsifiers) is prepared by adding the emulsifiers to hot water and stirring to dissolve. To the resulting solution is added hydroxypropylmethyl cellulose and Agaroid® 300 (agar and locust bean gum mixture), followed by mixing. To the thickened solution is added glycerin followed by the remaining ingredients. Mixing is continued until smooth. The resulting slurry was cast onto a flat carrier and dried to about 3% moisture then cut into strips about 11 mm by 32 mm size and about 140 mg in weight. Optionally, the cut strip may be perforated to enable the portioning of each strip. Each strip contains 0.5 mg of 2-methyl-5-(1-methylpyrrolidin-2-yl)pyridine.

TABLE 23
Composition ingredients

| Ingredient | % w/w |
|---|---|
| Water | 70-80 |
| 2-Methyl-5-(1-methylpyrrolidin-2-yl)pyridine free base | 0.8-1.5 |
| (12% aq. solution) | |
| Glyceryl monostearate | 1-2 |
| Polyoxy1 40 stearate | 2-3 |
| MCC | 1-3 |
| Hydroxypropylmethyl cellulose | 10-15 |
| Agaroid ® 300 | 0.4-0.6 |
| Glycerin | 6-8 |
| Modified food starch | 1-2 |
| Sweetener | 2-4 |
| Sodium chloride | 0.1-0.4 |
| Sodium benzoate | 0.3-1.0 |
| Flavor | 0-2 |

### Example 22. Preparation of Oral Products with Additional Active

Oral products in the form of a pouch, gum, tablet, chew, melt, pastille, lozenge or strip is prepared as in any of Examples 1-21, but in each instance, further including an α7 nicotinic receptor subtype (α7 NAChR) agonist. In each instance, the product is prepared as described in any of Examples 1-21, having a composition as described in any of Tables 2-21 or 23, and including from 0.2 mg to 5 mg of 2-methyl-5-(1-methylpyrrolidin-2-yl)pyridine and from about 0.1 to about 350 mg of the α7 NAChR agonist.

### Example 23. Preparation of a Pouched Oral Product (Slim Size, Sweet)

A pouched oral product comprising a fill composition according to the present disclosure was prepared. The composition included 2-methyl-5-(1-methylpyrrolidin-2-yl)pyridine as the active ingredient, with each pouch comprising 0.25 mg to 3 mg of the 2-methyl-5-(1-methylpyrrolidin-2-yl)pyridine. The composition further included microcrystalline cellulose (MCC), sweeteners, water, and additional components as disclosed herein (salt, buffer, humectant, flavoring agent). The fill composition had the formulation provided in Table 24. To prepare the composition, a blend of 2-methyl-5-(1-methylpyrrolidin-2-yl)pyridine (aqueous solution of benzoate salt, 11% by weight), water, MCC, salt, sodium benzoate, sodium bicarbonate (4% aqueous solution), propylene glycol, sodium citrate solution (about pH 6.3), sweeteners and flavoring agent was mixed in a blender. Portions of the fill composition (about 440 mg) were placed in fleece pouches (about 38 mm by 12 mm). Additional water was added to the pouches to achieve a pouch weight of about 720 mg and calculated oven volatiles content of about 48% by weight. The pouch pH is about pH 6.

TABLE 24
Oral product composition ingredients

| Ingredient | % w/w |
|---|---|
| microcrystalline cellulose | 65-75 |
| water | 0.8-6 |
| 2-methyl-5-(1-methylpyrrolidin-2- | 0.5-6.1 |
| yl)pyridine benzoate, 11% aq. solution | |
| sweetener | 0.5-5 |
| sodium chloride | 3.5-4.2 |
| sodium bicarbonate (4% aq) | 0.7-1.1 |
| propylene glycol | 8-9 |
| xylitol | |
| sodium benzoate | 0.1-1.2 |
| 0.5M sodium citrate solution | 2-2.5 |
| Flavor agent | 0-3 |

### Example 24. Preparation of a Pouched Oral Product (Slim Size, Less Sweet)

A pouched oral product comprising a fill composition according to the present disclosure was prepared. The composition included 2-methyl-5-(1-methylpyrrolidin-2-yl)pyridine as the active ingredient, with each pouch comprising 0.25 mg to 3 mg of the 2-methyl-5-(1-methylpyrrolidin-2-yl)pyridine. The composition further included microcrystalline cellulose (MCC), sweeteners, water, and additional components as disclosed herein (salt, buffer, humectant, flavoring agent). The fill composition had the formulation provided in Table 25. To prepare the composition, a blend of 2-methyl-5-(1-methylpyrrolidin-2-yl)pyridine (aqueous solution of benzoate salt, 11% by weight), water, MCC, salt, sodium benzoate, sodium bicarbonate solution, propylene glycol, sodium citrate solution (about pH 6.3), sweeteners and flavoring agent was mixed in a blender. Portions of the fill composition (about 440 mg) were placed in fleece pouches (37.5 mm by 12 mm). Additional water was added to the pouches to achieve a pouch weight of about 720 mg and calculated oven volatiles content of about 48% by weight. The pouch pH is about pH 6.

TABLE 25
Oral product fill composition ingredients

| Ingredient | % w/w |
|---|---|
| microcrystalline cellulose | 65-75 |
| water | 0.8-6 |
| 2-methyl-5-(1-methylpyrrolidin-2-yl)pyridine benzoate, | 0.5-6.1 |
| 11% aq. solution | |
| sweetener | 0.2-5 |
| sodium chloride | 4.5-5.5 |
| sodium bicarbonate (4% aq) | 0.7-1.5 |
| propylene glycol | 8.5-9 |
| sodium benzoate | 0.1-1.2 |
| 0.5M citrate solution | 2-2.5 |
| flavoring agent | 0-3 |

### Example 25. Preparation of a Pouched Oral Product (Mini Size, Sweet)

A pouched oral product comprising a fill composition according to the present disclosure was prepared. The composition included 2-methyl-5-(1-methylpyrrolidin-2-yl)pyridine as the active ingredient, with each pouch comprising 0.25 mg to 2 mg of the 2-methyl-5-(1-methylpyrrolidin-2-yl)pyridine. The composition further included microcrystalline cellulose (MCC), sweeteners, water, and additional components as disclosed herein (salt, buffer, humectant, flavoring agent). The fill composition had the formulation provided in Table 26. To prepare the composition, a blend of 2-methyl-5-(1-methylpyrrolidin-2-yl)pyridine (aqueous solution of benzoate salt, 11% by weight), water, MCC, salt, sodium benzoate, sodium bicarbonate solution, propylene glycol, sodium citrate solution (about pH 6.3), sweeteners and flavoring agent was mixed in a blender. Portions of the fill composition (about 323 mg) were placed in fleece pouches (about 30 mm by 12 mm). Additional water was added to the pouches to achieve a pouch weight of about 426 mg and a calculated oven volatiles content of about 35% by weight). The pouch pH is about pH 6.

TABLE 26
Oral product fill composition ingredients

| Ingredient | % w/w |
|---|---|
| microcrystalline cellulose | 65-75 |
| water | 0.1-5 |
| 2-methyl-5-(1-methylpyrrolidin-2-yl)pyridine benzoate, | 0.7-6 |
| 11% aq. solution | |
| sweetener | 0.5-5 |
| sodium chloride | 3.5-4.2 |
| sodium bicarbonate (4% aq) | 1-1.5 |
| propylene glycol | 8-9 |
| sodium benzoate | 0.1-1.2 |
| 0.5M citrate solution | 2-3 |
| flavoring agent | 0-4 |

### Example 26. Preparation of a Pouched Oral Product (Mini Size, Less Sweet)

A pouched oral product comprising a fill composition according to the present disclosure was prepared. The composition included 2-methyl-5-(-methylpyrrolidin-2-yl)pyridine as the active ingredient, with each pouch comprising 0.25 mg to 2 mg of the 2-menthyl-5-(1-˜methylpyrrolidin-2-yl)pyridine. The composition further included microcrystalline cellulose (MCC), sweeteners, water, and additional components as disclosed herein (salt, buffer, humectant, flavoring agent). The fill composition had the formulation provided in Table 27. To prepare the composition, a blend of 2-methyl-5-(1-methylpyrrolidin-2-yl)pyridine (aqueous solution of benzoate salt, 11% by weight), water, MCC, salt, sodium benzoate, sodium bicarbonate solution, propylene glycol, sodium citrate solution (about pH 6.3), sweeteners and flavoring agent was mixed in a blender. Portions of the composition (about 323 mg) were placed in fleece pouches (about 30 mm by 12 mm). Additional water was added to the pouches to achieve a pouch weight of about 426 mg and a calculated oven volatiles content of about 35% by weight). The pouch pH is about pH 6.

TABLE 27
Oral product fill composition ingredients

| Ingredient | % w/w |
|---|---|
| microcrystalline cellulose | 65-75 |
| water | 0.1-5 |
| 2-methyl-5-(1-methylpyrrolidin-2-yl)pyridine benzoate, | 0.7-6 |
| 11% aq. solution | |
| sweetener | 0.2-5 |
| sodium chloride | 4.5-5.5 |
| sodium bicarbonate (4% aq) | 1-1.5 |
| propylene glycol | 8-9 |
| sodium benzoate | 0.1-1.2 |
| 0.5M citrate solution | 2-3 |
| flavoring agent | 0-3.5 |

### Example 27. Preparation of a High pH Dip

An oral product in dip (“snuff”) form comprising a composition according to the present disclosure is prepared. The composition includes 2-methyl-5-(1-methylpyrrolidin-2-yl)pyridine as the active ingredient, with each 2 g portion comprising from 0.1 mg to 1.5 mg of the 2-methyl-5-(1-methylpyrrolidin-2-yl)pyridine. The composition further includes a non-tobacco botanical material, sweetener, water, and additional components as disclosed herein (salt, buffer, humectants, flavoring agent, preservatives, colorant). The composition has the formulation provided in Table 28. To prepare the composition, 2-methyl-5-(1-methylpyrrolidin-2-yl)pyridine aqueous solution (12% by weight), water, botanical material, salt, sodium benzoate, sodium bicarbonate, humectant, sweetener, flavoring agent, colorant, and preservative are mixed in a blender. The mixture is portioned into approximately 2 g quantities. The final moisture content is about 50%. The composition, when placed in water, has a pH in a range from about 7 to about 9. In some embodiments, the composition (about 1.3 g) is placed in a cellulosic fleece to form a pouched product.

TABLE 28
High pH oral product composition ingredients

| Ingredient | % w/w |
|---|---|
| non-tobacco material | 16-50 |
| water | 8-50 |
| 2-methyl-5-(1-methylpyrrolidin-2-yl)pyridine | 0.04-0.6 |
| (12% aqueous) | |
| sweetener | 0.2-0.5 |
| sodium chloride | 2-9 |
| humectant | 5-30 |
| sodium benzoate | 0-1.64 |
| sodium bicarbonate | 0-3 |
| flavor | 0-4 |
| coloring | 0-8 |
| preservative | 0-2 |

### Example 28. Preparation of a Low pH Dip

An oral product in dip (“snuff”) form comprising a composition according to the present disclosure is prepared. The composition includes 2-methyl-5-(1-methylpyrrolidin-2-yl)pyridine as the active ingredient, with each 2 g portion comprising from 0.1 mg to 1.5 mg of the 2-methyl-5-(1-methylpyrrolidin-2-yl)pyridine. The composition further includes a non-tobacco botanical material, sweetener, water, and additional components as disclosed herein (salt, buffer, humectants, flavoring agent, preservatives, colorant). The composition has the formulation provided in Table 29. To prepare the composition, 2-methyl-5-(1-methylpyrrolidin-2-yl)pyridine benzoate aqueous solution (11% by weight), water, botanical material, salt, sodium benzoate, sodium bicarbonate, humectant, sweetener, flavoring agent, colorant, and preservative are mixed in a blender. The mixture is portioned into approximately 2 g quantities. The final moisture content is about 50%. The composition, when placed in water, has a pH in a range from about 5 to about 7. In some embodiments, the composition (about 1.3 g) is placed in a cellulosic fleece to form a pouched product.

TABLE 29
Low pH oral product composition ingredients

| Ingredient | % w/w |
|---|---|
| non-tobacco material | 16-50 |
| water | 8-50 |
| 2-methyl-5-(1-methylpyrrolidin-2-yl)pyridine | 0.05-0.7 |
| benzoate (11% aqueous) | |
| sweetener | 0.2-0.5 |
| sodium chloride | 2-9 |
| humectant | 5-30 |
| sodium benzoate | 0-1.22 |
| sodium bicarbonate | 0-1 |
| flavor | 0-4 |
| Coloring | 0-8 |
| Preservative | 0-2 |

### Example 29. Preparation of an Effervescent Tablet

Effervescent tablets according to embodiments of the disclosure are prepared. A composition in tablet form is prepared comprising a mixture of fillers, about 0.25 mg to 2 mg 2-methyl-5-(1-methylpyrrolidin-2-yl)pyridine, and additional components as disclosed herein (salt, sweeteners, sodium carbonate, sodium bicarbonate, citric acid, processing aid). Aqueous 2-methyl-5-(1-methylpyrrolidin-2-yl)pyridine benzoate (aqueous, 11% by weight) and microcrystalline cellulose are mixed. The fillers, sweetener, salt, 2-methyl-5-(1-methylpyrrolidin-2-yl)pyridine benzoate microcrystalline cellulose mixture, sodium carbonate, sodium bicarbonate, citric acid, and processing aid are combined and mixed thoroughly. The mixture is compressed and tableted using a Fette 1200i tablet press. The tablets are optionally coated with wax and shellac. The ingredients of the composition and their concentrations in the composition in weight % are provided in Table 30. The tablets each weigh 800 mg.

TABLE 30
Effervescent tablet composition ingredients

| Ingredient | % w/w |
|---|---|
| Emdex ® | 50-61 |
| Isomalt | 19-23 |
| Hydroxypropyl Cellulose | 3-4 |
| Sweetener | 0.1-1 |
| Sodium Chloride | 0.25-0.75 |
| Sodium Benzoate | 5.9-7.2 |
| Flavor | 0-1 |
| Sodium Carbonate | 0.9-1.1 |
| Sodium Bicarbonate | 5.9-7.2 |
| Citric Acid | 4.5-5.5 |
| Colorant | 0-0.3 |
| Sodium stearyl fumarate | 1.1-1.4 |
| 2-methyl-5-(1-methylpyrrolidin-2-yl)pyridine | 0.3-2.3 |
| benzoate (11% aqueous) | |
| Microcrystalline cellulose | 0.5-8 |

### Example 30. Long-Term and Accelerated Storage Stability Studies

Pouched oral products according to embodiments of the disclosure, prepared according to Examples 8, 9, 16, 17, 18, and 19) were evaluated for storage stability under both long term and accelerated conditions in a stability chamber. The pouches were packed in conventional oral product containers with side seam seals (20 pouches/can) for the stability testing. The conditions for long-term storage (ambient) were a temperature of 25° C.±2° C. and relative humidity of 60% ±5%. The conditions for accelerated storage were a temperature of 40° C.±2° C. and relative humidity of 75% ±5%. Results are provided in Tables 31 and 32 for long-term and accelerated conditions, respectively.

With reference to Table 31, it was observed that there was typically a difference in pH between the fill material and the pouched product which varied with e.g., pouch size, concentration of 2-methyl-5-(1-methylpyrrolidin-2-yl)pyridine, and/or moisture content. The difference between the fill pH and the pouch pH provided in Table 31 show that the pH difference increased as the concentration of compound in the fill composition decreased. With continued reference to Table 31, the data demonstrate that the initial pouch pH value of (approximately 6) for the various pouched products of the disclosure was maintained over the duration of the six-month storage period under ambient conditions. Notably, these compositions did not include a citrate buffer.

Similarly, with continued reference to Table 31, the initial moisture content of the various pouched products of the disclosure (i.e., either approximately 35% or approximately 48% moisture content) was maintained over the duration of the six-month storage period under ambient conditions. Finally, with continued reference to Table 31, the initial concentration of 2-methyl-5-(1-methylpyrrolidin-2-yl)pyridine (approximately 1, 3, or 5 mg per pouch). was maintained over the duration of the six-month storage period under ambient conditions.

With reference to Table 32, the data demonstrate that the initial pH value, moisture content, and concentration of 2-methyl-5-(1-methylpyrrolidin-2-yl)pyridine for the various pouched products of the disclosure were all maintained over the duration of the six-month storage period under accelerated aging conditions.

Together, the results in Tables 31 and 32 indicate that products of the disclosure comprising 2-methyl-5-(1-methylpyrrolidin-2-yl)pyridine were surprisingly stable over the storage period with respect to pH, moisture content, and concentration of 2-methyl-5-(1-methylpyrrolidin-2-yl)pyridine. Particularly surprising was the retention over time of the initial concentration of 2-methyl-5-(1-methylpyrrolidin-2-yl)pyridine. This was unexpected in view of the very small amount present per pouch and the propensity of comparable nicotine pouches to exhibit a reduction in nicotine concentration over time.

Accordingly, in some embodiments, a pouched product of the disclosure comprising a substituted 3-(1-methylpyrrolidin-2-yl)pyridine (e.g., 2-methyl-5-(1-methylpyrrolidin-2-yl)pyridine) maintains one or more of the following when stored at a temperature of 25° C.±2° C. and relative humidity of 60% ±5% for a period of up to 6 months:
a concentration of a substituted 3-(1-methylpyrrolidin-2-yl)pyridine (e.g., 2-methyl-5-(1-methylpyrrolidin-2-yl)pyridine) which is within ±10% of an initial concentration of the substituted 3-(1-methylpyrrolidin-2-yl)pyridine (e.g., 2-methyl-5-(1-methylpyrrolidin-2-yl)pyridine), wherein the initial concentration is about 1 mg, about 3 mg, or about 5 mg per pouch;
a moisture content which is within ±5% of an initial moisture content, wherein the initial moisture content is about 35% or about 48%;
a pH value which is within ±10% of an initial pH value, wherein the initial pH value is about 6.

In some embodiments, a pouched product of the disclosure comprising a substituted 3-(1-methylpyrrolidin-2-yl)pyridine (e.g., 2-methyl-5-(1-methylpyrrolidin-2-yl)pyridine) maintains one or more of the following when stored at a temperature of 40° C.±2° C. and relative humidity of 75% ±5% for a period of up to 6 months:
a concentration of a substituted 3-(1-methylpyrrolidin-2-yl)pyridine (e.g., 2-methyl-5-(1-methylpyrrolidin-2-yl)pyridine) which is within ±10% of an initial concentration of the substituted 3-(1-methylpyrrolidin-2-yl)pyridine (e.g., 2-methyl-5-(1-methylpyrrolidin-2-yl)pyridine), wherein the initial concentration is about 1 mg, about 3 mg, or about 5 mg per pouch;
a moisture content which is within ±5% of an initial moisture content, wherein the initial moisture content is about 35% or about 48%;
a pH value which is within ±10% of an initial pH value, wherein the initial pH value is about 6.

TABLE 31
Long-term storage (ambient)
0 Months (Initial)

| | Description | | | | | | | Fill |
|---|---|---|---|---|---|---|---|---|
| | (mg active*, | | | | | | | pH- |
| | moisture | active | active | Weight | Moisture | Pouch | Fill | Pouch |
| Ex. # | content**, flavor) | (mg/g) | (mg/pouch) | (g) | (%) | pH | pH | pH |
| 9 | 1 mg, 35%, | 1.68 | 0.95 | 0.564 | 33.7 | 6.04 | 7.36 | 1.32 |
| | unflavored | | | | | | | |
| 9 | 1 mg, 35%, | 1.68 | 0.98 | 0.581 | 33.1 | 6.01 | 7.42 | 1.41 |
| | flavored | | | | | | | |
| 9 | 3 mg, 35%, | 5.15 | 2.84 | 0.551 | 33.6 | 6.01 | 6.7 | 0.69 |
| | unflavored | | | | | | | |
| 9 | 3 mg, 35%, | 5.11 | 2.87 | 0.562 | 32.8 | 5.97 | 6.65 | 0.68 |
| | flavored | | | | | | | |
| 8 | 5 mg, 35%, | 8.66 | 4.98 | 0.575 | 33.3 | 5.92 | 6.29 | 0.37 |
| | flavored | | | | | | | |
| 17 | 1 mg, 35%, | 2.32 | 0.99 | 0.428 | 32.1 | 6.35 | 7.74 | 1.39 |
| | flavored | | | | | | | |
| 18 | 3 mg, 35%, | 6.97 | 2.93 | 0.42 | 31.9 | 6.13 | 6.97 | 0.84 |
| | flavored | | | | | | | |
| 16 | 1 mg, 48%, | 1.28 | 0.95 | 0.737 | 47.4 | 6.09 | 7.36 | 1.27 |
| | unflavored | | | | | | | |
| 16 | 1 mg, 48%, | 1.42 | 0.98 | 0.690 | 43.6 | 6.03 | 7.42 | 1.39 |
| | flavored | | | | | | | |
| 9 | 3 mg, 48%, | 3.91 | 2.79 | 0.714 | 48.0 | 6.03 | 6.70 | 0.67 |
| | unflavored | | | | | | | |
| 9 | 3 mg, 48%, | 4.18 | 2.98 | 0.713 | 45.2 | 5.98 | 6.65 | 0.67 |
| | flavored | | | | | | | |
| 8 | 5 mg, | 6.67 | 4.72 | 0.708 | 46.4 | 5.93 | 6.29 | 0.36 |
| | 48%, | | | | | | | |
| | flavored | | | | | | | |
| 19 | 1 mg, 48%, | 1.35 | 0.98 | 0.725 | 44.8 | 6.04 | 7.12 | 1.08 |
| | flavored | | | | | | | |

3 Months

| | Description | | | | | |
|---|---|---|---|---|---|---|
| | (mg active*, | | | | | |
| | moisture | active | active | Weight | Moisture | |
| Example # | content**, flavor) | (mg/g) | (mg/pouch) | (g) | (%) | pH |
| 9 | 1 mg, 35%, | 1.84 | 1.06 | 0.574 | 31.9 | 6.05 |
| | unflavored | | | | | |
| 9 | 1 mg, 35%, | 1.8 | 1.05 | 0.586 | 33.7 | 6.02 |
| | flavored | | | | | |
| 9 | 3 mg, 35%, | 5.56 | 3.11 | 0.560 | 32.4 | 5.99 |
| | unflavored | | | | | |
| 9 | 3 mg, 35%, | 5.56 | 3.17 | 0.570 | 32.8 | 5.97 |
| | flavored | | | | | |
| 8 | 5 mg, 35%, | 9.26 | 5.31 | 0.573 | 33.3 | 5.93 |
| | flavored | | | | | |
| 17 | 1 mg, 35%, | 2.48 | 1.04 | 0.419 | 31.6 | 6.37 |
| | flavored | | | | | |
| 18 | 3 mg, 35%, | 7.48 | 3.11 | 0.416 | 31.4 | 6.23 |
| | flavored | | | | | |
| 16 | 1 mg, 48%, | 1.43 | 1.01 | 0.709 | 47.1 | 6.08 |
| | unflavored | | | | | |
| 16 | 1 mg, 48%, | 1.53 | 1.07 | 0.698 | 44.9 | 6.05 |
| | flavored | | | | | |
| 9 | 3 mg, 48%, | 4.12 | 2.90 | 0.705 | 48.2 | 6.00 |
| | unflavored | | | | | |
| 9 | 3 mg, 48%, | 4.61 | 3.30 | 0.715 | 44.3 | 5.98 |
| | flavored | | | | | |
| 8 | 5 mg, 48%, | 7.38 | 5.16 | 0.699 | 46.5 | 5.97 |
| | flavored | | | | | |
| 19 | 1 mg, 48%, | 1.44 | 1.05 | 0.727 | 44.5 | 5.92 |
| | flavored | | | | | |

6 Months

| | Description | | | | | |
|---|---|---|---|---|---|---|
| | (mg active*, | | | | | |
| | moisture | active | active | Weight | Moisture | |
| Example # | content**, flavor) | (mg/g) | (mg/pouch) | (g) | (%) | pH |
| 9 | 1 mg, 35%, | 1.85 | 1.03 | 0.558 | 31.3 | 5.95 |
| | unflavored | | | | | |
| 9 | 1 mg, 35%, | 1.85 | 1.05 | 0.568 | 32.2 | 6.06 |
| | flavored | | | | | |
| 9 | 3 mg, 35%, | 5.55 | 3.01 | 0.542 | 32.6 | 5.96 |
| | unflavored | | | | | |
| 9 | 3 mg, 35%, | 5.50 | 3.10 | 0.563 | 32.4 | 6.06 |
| | flavored | | | | | |
| 8 | 5 mg, 35%, | 9.1 | 5.15 | 0.566 | 33.1 | 5.95 |
| | flavored | | | | | |
| 17 | 1 mg, 35%, | 2.51 | 1.04 | 0.415 | 31.8 | 6.35 |
| | flavored | | | | | |
| 18 | 3 mg, 35%, | 7.44 | 3.08 | 0.414 | 31.7 | 6.20 |
| | flavored | | | | | |
| 16 | 1 mg, 48%, | 1.47 | 1.06 | 0.723 | 46.4 | 5.97 |
| | unflavored | | | | | |
| 16 | 1 mg, 48%, | 1.51 | 1.05 | 0.694 | 44.9 | 6.06 |
| | flavored | | | | | |
| 9 | 3 mg, 48%, | 4.26 | 2.97 | 0.698 | 47.9 | 6.15 |
| | unflavored | | | | | |
| 9 | 3 mg, | 4.49 | 3.20 | 0.714 | 45.3 | 6.06 |
| | 48%, | | | | | |
| | flavored | | | | | |
| 8 | 5 mg, 48%, | 7.34 | 5.17 | 0.704 | 46.6 | 5.99 |
| | flavored | | | | | |
| 19 | 1 mg, 48%, | 1.53 | 1.09 | 0.713 | 44.5 | 5.99 |
| | flavored | | | | | |

*active refers to 2-methyl-5-(1-methylpyrrolidin-2-yl)pyridine
** = moisture content as calculated loss on drying (%)
Unflavored = 0% flavor agent

TABLE 32
Long-term storage (Accelerated)
0 Months (Initial)

| | Description (mg active*, | | | | | |
|---|---|---|---|---|---|---|
| Example | moisture content **, | active | active | Weight | | |
| # | flavor) | (mg/g) | (mg/pouch) | (g) | Moisture (%) | pH |
| 9 | 1 mg, 35%, unflavored | 1.68 | 0.95 | 0.564 | 33.69 | 6.04 |
| 9 | 1 mg, 35%, flavored | 1.68 | 0.98 | 0.581 | 33.13 | 6.01 |
| 9 | 3 mg, 35%, unflavored | 5.15 | 2.84 | 0.551 | 33.58 | 6.01 |
| 9 | 3 mg, 35%, flavored | 5.11 | 2.87 | 0.562 | 32.75 | 5.97 |
| 8 | 5 mg, 35%, flavored | 8.66 | 4.98 | 0.575 | 33.34 | 5.92 |
| 17 | 1 mg, 35%, flavored | 2.32 | 0.99 | 0.428 | 32.14 | 6.35 |
| 18 | 3 mg, 35%, flavored | 6.97 | 2.93 | 0.42 | 31.95 | 6.13 |
| 16 | 1 mg, 48%, unflavored | 1.28 | 0.95 | 0.737 | 47.37 | 6.09 |
| 16 | 1 mg, 48%, flavored | 1.42 | 0.98 | 0.690 | 43.57 | 6.03 |
| 9 | 3 mg, 48%, unflavored | 3.91 | 2.79 | 0.714 | 48.00 | 6.03 |
| 9 | 3 mg, 48%, flavored | 4.18 | 2.98 | 0.713 | 45.23 | 5.98 |
| 8 | 5 mg, 48%, flavored | 6.67 | 4.72 | 0.708 | 46.41 | 5.93 |
| 19 | 1 mg, 48%, flavored | 1.35 | 0.98 | 0.725 | 44.77 | 6.04 |

1 Month

| | Description (mg active*, | | | | | |
|---|---|---|---|---|---|---|
| Example | moisture content**, | active | active | Weight | | |
| # | flavor) | (mg/g) | (mg/pouch) | (g) | Moisture (%) | pH |
| 9 | 1 mg, 35%, unflavored | 1.71 | 0.97 | 0.568 | 33.66 | 6.13 |
| 9 | 1 mg, 35%, flavored | 1.73 | 1.00 | 0.579 | 32.87 | 5.96 |
| 9 | 3 mg, 35%, unflavored | 5.21 | 2.88 | 0.554 | 33.17 | 6.13 |
| 9 | 3 mg, 35%, flavored | 5.23 | 2.95 | 0.563 | 32.61 | 5.98 |
| 8 | 5 mg, 35%, flavored | 8.66 | 4.98 | 0.575 | 33.50 | 5.94 |
| 17 | 1 mg, 35%, flavored | 2.39 | 1.01 | 0.422 | 31.61 | 6.30 |
| 18 | 3 mg, 35%, flavored | 0 | 2.96 | 0.415 | 31.98 | 6.21 |
| 16 | 1 mg, 48%, unflavored | 1.37 | 1.00 | 0.726 | 45.94 | 6.13 |
| 16 | 1 mg, 48%, flavored | 1.38 | 0.98 | 0.710 | 44.53 | 5.99 |
| 9 | 3 mg, 48%, unflavored | 3.96 | 2.79 | 0.705 | 48.50 | 6.12 |
| 9 | 3 mg, 48%, flavored | 4.2 | 3.00 | 0.714 | 45.01 | 6.12 |
| 8 | 5 mg, 48%, flavored | 6.95 | 5.04 | 0.725 | 46.57 | 5.97 |
| 19 | 1 mg, 48%, flavored | 1.42 | 1.05 | 0.742 | 44.62 | 5.94 |

2 Months

| | Description (mg active*, | | | | | |
|---|---|---|---|---|---|---|
| Example | moisture content**, | active | active | Weight | | |
| # | flavor) | (mg/g) | (mg/pouch) | (g) | Moisture (%) | pH |
| 9 | 1 mg, 35%, unflavored | 1.65 | 0.92 | 0.559 | 33.11 | 5.93 |
| 9 | 1 mg, 35%, flavored | 1.66 | 0.96 | 0.577 | 32.61 | 5.91 |
| 9 | 3 mg, 35%, unflavored | 5.03 | 2.80 | 0.556 | 32.38 | 5.97 |
| 9 | 3 mg, 35%, flavored | 5.09 | 2.82 | 0.553 | 31.99 | 5.93 |
| 8 | 5 mg, 35%, flavored | 8.26 | 4.78 | 0.578 | 33.50 | 5.91 |
| 17 | 1 mg, 35%, flavored | 2.28 | 0.94 | 0.414 | 31.46 | 6.24 |
| 18 | 3 mg, 35%, flavored | 6.7 | 2.77 | 0.413 | 31.51 | 6.16 |
| 16 | 1 mg, 48%, unflavored | 1.36 | 0.98 | 0.722 | 45.71 | 5.96 |
| 16 | 1 mg, 48%, flavored | 1.36 | 0.96 | 0.709 | 44.98 | 5.92 |
| 9 | 3 mg, 48%, unflavored | 3.91 | 2.77 | 0.708 | 47.93 | 6.02 |
| 9 | 3 mg, 48%, flavored | 4.21 | 3.05 | 0.725 | 44.91 | 5.97 |
| 8 | 5 mg, 48%, flavored | 6.89 | 4.98 | 0.723 | 45.65 | 5.97 |
| 19 | 1 mg, 48%, flavored | 1.37 | 1.00 | 0.726 | 44.62 | 5.94 |

3 Months

| | Description (mg active*, | | | | | |
|---|---|---|---|---|---|---|
| Example | moisture content **, | active | active | Weight | | |
| # | flavor) | (mg/g) | (mg/pouch) | (g) | Moisture (%) | pH |
| 9 | 1 mg, 35%, unflavored | 1.81 | 1.02 | 0.562 | 32.91 | 6.10 |
| 9 | 1 mg, 35%, flavored | 1.79 | 1.03 | 0.576 | 32.56 | 5.94 |
| 9 | 3 mg, 35%, unflavored | 5.46 | 2.99 | 0.547 | 32.24 | 6.07 |
| 9 | 3 mg, 35%, flavored | 5.47 | 3.09 | 0.564 | 32.98 | 5.95 |
| 8 | 5 mg, 35%, flavored | 9.19 | 5.24 | 0.570 | 33.28 | 5.93 |
| 17 | 1 mg, 35%, flavored | 2.41 | 1.01 | 0.418 | 31.07 | 6.22 |
| 18 | 3 mg, 35%, flavored | 7.42 | 3.09 | 0.416 | 30.99 | 6.17 |
| 16 | 1 mg, 48%, unflavored | 1.44 | 1.05 | 0.729 | 44.92 | 6.11 |
| 16 | 1 mg, 48%, flavored | 1.45 | 1.02 | 0.706 | 44.88 | 5.99 |
| 9 | 3 mg, 48%, unflavored | 4.19 | 2.96 | 0.706 | 47.88 | 6.11 |
| 9 | 3 mg, 48%, flavored | 4.53 | 3.15 | 0.696 | 44.10 | 6.01 |
| 8 | 5 mg, 48%, flavored | 7.34 | 5.20 | 0.708 | 46.36 | 5.98 |
| 19 | 1 mg, 48%, flavored | 1.46 | 1.06 | 0.727 | 44.34 | 5.92 |

6 Months

| | Description (mg active*, | | | | | |
|---|---|---|---|---|---|---|
| Example | moisture content **, | active | active | Weight | | |
| # | flavor) | (mg/g) | (mg/pouch) | (g) | Moisture (%) | pH |
| 9 | 1 mg, 35%, unflavored | 1.83 | 1.04 | 0.569 | 32.36 | 5.94 |
| 9 | 1 mg, 35%, flavored | 1.82 | 1.04 | 0.572 | 31.43 | 5.95 |
| 9 | 3 mg, 35%, unflavored | 5.46 | 3.02 | 0.554 | 32.16 | 5.92 |
| 9 | 3 mg, 35%, flavored | 5.53 | 3.10 | 0.560 | 31.32 | 5.91 |
| 8 | 5 mg, 35%, flavored | 9.08 | 5.13 | 0.565 | 32.16 | 5.90 |
| 17 | 1 mg, 35%, flavored | 2.50 | 1.03 | 0.413 | 30.61 | 6.14 |
| 18 | 3 mg, 35%, flavored | 7.48 | 3.05 | 0.408 | 29.98 | 6.08 |
| 16 | 1 mg, 48%, unflavored | 1.50 | 1.08 | 0.715 | 45.53 | 6.00 |
| 16 | 1 mg, 48%, flavored | 1.50 | 1.04 | 0.694 | 44.45 | 5.97 |
| 9 | 3 mg, 48%, unflavored | 4.13 | 2.94 | 0.712 | 47.55 | 5.98 |
| 9 | 3 mg, 48%, flavored | 4.60 | 3.23 | 0.702 | 42.82 | 5.97 |
| 8 | 5 mg, 48%, flavored | 7.31 | 5.08 | 0.695 | 45.32 | 5.94 |
| 19 | 1 mg, 48%, flavored | 1.52 | 1.09 | 0.719 | 44.17 | 5.91 |

*active refers to 2-methyl-5-(1-methylpyrrolidin-2-yl)pyridine
**moisture content as calculated loss on drying (%)
Unflavored = 0% flavor agent

1. A composition configured for oral use, the composition comprising:
a filler component;
water in an amount from about 1 to about 60% by weight, based on the total weight of the composition; and
a substituted 3-(1-methylpyrrolidin-2-yl)pyridine having a structure according to Formula I: 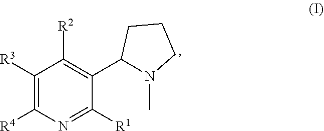
wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and
at least one of R¹, R², R³, and R⁴ are not hydrogen.

2. The composition of claim 1, wherein R¹, R², and R³ are each H.

3. The composition of claim 1, wherein R⁴ is optionally substituted C₁-C₆ alkyl, F, Cl, Br, OCH₃, OEt, or CN.

4. The composition of claim 1, wherein R¹, R², and R³ are each H, and R⁴ is C₁-C₃ alkyl; preferably, wherein R⁴ is CH₃.

5. The composition of claim 1, wherein the substituted 3-(1-methylpyrrolidin-2-yl)pyridine is racemic.

6. The composition of claim 1, wherein the substituted 3-(1-methylpyrrolidin-2-yl)pyridine has an (R)-configuration or an (S)-configuration; preferably, wherein the substituted 3-(1-methylpyrrolidin-2-yl)pyridine has an (S)-configuration.

7. The composition of claim 1, wherein the substituted 3-(1-methylpyrrolidin-2-yl)pyridine has a calculated log P of about 1 or greater, such as from about 1.2 to about 1.7.

8. The composition of claim 1, wherein the composition is substantially free of 3-(1-methylpyrrolidin-2-yl)pyridine; preferably, wherein the composition is completely free of 3-(1-methylpyrrolidin-2-yl)pyridine.

9. The composition of claim 1, wherein the composition is substantially free of acid components; optionally, wherein the composition is completely free of acid components.

10. The composition of claim 1, further comprising an organic acid, an alkali metal salt of an organic acid, or a combination thereof, the organic acid having a log P value in a range from about 1 to about 12; optionally, wherein the organic acid has a log P value from about 3 to about 12, from about 3 to about 10, or from about 3 to about 8, and wherein at least a portion of the substituted 3-(1-methylpyrrolidin-2-yl)pyridine is present in the form of an ion pair with the organic acid, the alkali metal salt of an organic acid, or a combination thereof.

11. The composition of claim 1, wherein at least a portion of the substituted 3-(1-methylpyrrolidin-2-yl)pyridine is present in the form of a salt, a co-crystal, or a salt co-crystal.

12. The composition of claim 1, wherein at least a portion of the substituted 3-(1-methylpyrrolidin-2-yl)pyridine is bound to a polymeric resin.

13. The composition of claim 1, wherein the substituted 3-(1-methylpyrrolidin-2-yl)pyridine is present in an amount from about 0.01 to about 10% by weight of the composition, calculated as the free base and based on the total weight of the composition.

14. The composition of claim 1, wherein the filler component is cellulosic; optionally, wherein the filler component comprises microcrystalline cellulose.

15. The composition of claim 1, further comprising one or more flavoring agents, one or more salts, one or more sweeteners, one or more binding agents, one or more humectants, one or more gums, or combinations thereof.

16. The composition of claim 1, in the form of a gel, pastille, gum, chew, melt, tablet, lozenge, film, granular material, snuff, or powder.

17. The composition of claim 1, enclosed in a pouch to form a pouched product.

18. The composition of claim 17, having a moisture content from about 30 to about 60% by weight, based on the weight of the pouched product.

19. A composition configured for oral use, the composition comprising:
2-methyl-5-(1-methylpyrrolidin-2-yl)pyridine in an amount from about 0.01 to about 10% by weight, based on the total weight of the composition;
a filler component; and
water in an amount from about 1 to about 60% by weight, or from about 30 to about 50% by weight, based on the total weight of the composition.

20. The composition of claim 19, wherein the filler component is cellulosic; optionally, wherein the filler component comprises microcrystalline cellulose.

21. The composition of claim 19, further comprising a buffer; optionally, wherein the buffer is an alkali metal citrate.

22. The composition of claim 19, wherein at least about 60% of a total amount of 2-methyl-5-(1-methylpyrrolidin-2-yl)pyridine by weight present in the composition is released when said composition is placed in 500 mL of phosphate buffer at pH 7.4, at 37° C. for 50 minutes in a basket-type USP dissolution apparatus at a stirring speed of 50 RPM.

23. The composition of claim 22, wherein at least about 20% of a total amount of 2-methyl-5-(1-methylpyrrolidin-2-yl)pyridine by weight present in the composition is released at 10 minutes.

24. A method of preparing a composition configured for oral use comprising a substituted 3-(1-methylpyrrolidin-2-yl)pyridine, the method comprising:
combining one or more fillers with one or more additional powder components, such as one or more salts and/or one or more sweeteners to form a dry blend;
combining a dilute aqueous solution of a substituted 3-(1-methylpyrrolidin-2-yl)pyridine with water and humectant to form a solution;
combining the solution with the dry blend to form a mixture;
mixing the resulting mixture to form a wet blend; and
optionally adding flavoring agent and/or water to the wet blend form the composition.

25. The method of claim 24, wherein the substituted 3-(1-methylpyrrolidin-2-yl)pyridine is 2-methyl-5-(1-methylpyrrolidin-2-yl)pyridine.
